(19) United States
(12) Patent Application Publication
(10) Pub. No.: US 2006/0008520 A1
(43) Pub. Date: Jan. 12, 2006

(54) Delayed release formulations of 6-mercaptopurine

(76) Inventors: E. Itzhak Lerner, Petach Tikva (IL); Moshe Flashner-Barak, Petach Tikva (IL); Erwin v Achthoven, Leiderdorp (NL); Hans Keegstra, Alkmaar (NL); Ruud Smit, Haarlem (NL)

Correspondence Address: KENYON & KENYON, ONE BROADWAY, NEW YORK, NY, 10004, US

(21) Appl. No.: 11/097,875

(22) Filed: Apr. 1, 2005

Related U.S. Application Data
(60) Provisional application No. 60/558,477, filed on Apr. 1, 2004.

(51) Int. Cl. A61K 9/48; A61K 9/22; A61K 31/522

(52) U.S. Cl. 424/451; 424/468; 514/263.3

(57) ABSTRACT

The present invention provides enterically coated formulations of 6-mercaptopurine that exhibit a delay in release of the 6-mercaptopurine such that substantial release of 6-mercaptopurine does not occur until after passage through the stomach. Optionally, the formulations also comprise a delay coating in addition to the enteric coating that provides an even further delay such that substantial release of 6-mercaptopurine does not occur until after a certain period of time following passage through the stomach. Such a period of time is preferably at least one hour after passage through the stomach. Following the delay imparted by the enteric coating and optional delay coating, the formulations exhibit better bioavailability and faster dissolution than previous formulations.

### CROSS-REFERENCE TO RELATED APPLICATIONS

This application claims the benefit of provisional application Ser. No. 60/558,447, filed Apr. 1, 2004, which is incorporated herein by reference.

### FIELD OF THE INVENTION

The present invention relates to a process for preparing improved formulations of 6-mercaptopurine as well as pharmaceutical compositions comprising the improved formulations of 6-mercaptopurine where the improved formulations exhibit a delayed release of 6-mercaptopurine such that 6-mercaptopurine is released after passage of the compositions through the stomach and into the intestine. Following the delayed release, the compositions may exhibit faster release of 6-mercaptopurine under aqueous conditions than prior art formulations and also may exhibit a more favorable bioavailability profiles than prior art formulations.

### BACKGROUND OF THE INVENTION

6-mercaptopurine (6-MP) is a synthetic analogue of natural purine bases. After absorption into the body, it is transformed into nucleotides which interfere with nucleic acid biosynthesis, especially in the active S phase. As such, it used to slow the growth of cancerous cells. 6-MP is indicated as a monotherapy and as part of combination therapies for treating acute lymphocytic leukemia in both adults and children (Physician's Desk Reference 57^{th} Edition, 2003, page 1615-1618). 6-MP also exhibits immunosuppressive properties. While it is not officially indicated for diseases where treatment with immunosuppressive agents is beneficial, 6-MP has been widely used for several such conditions, especially for Crohn's disease and colitis.

6-MP is administered orally and has partial and variable absorption and bioavailability. Approximately 50% of an oral dose is absorbed. 6-MP is further subject to metabolism, especially by thiopurine methyltransferase.

The need for improving the therapeutic potential of 6-MP has been known for a long time. U.S. Pat. Nos. 4,443,435 and 5,120,740, among others, describe the preparation of prodrugs for 6-MP as ways of improving the use of this potent drug. Work of this sort continues, as is seen in U.S. Patent Application Publications 20040013728, 20030232760, and 20020013287. U.S. Pat. Nos. 6,680,302; 6,576,438; and 6,355,623 describe methods of improving the therapeutic outcome of 6-MP treatment in leukemia and in bowel diseases such as Crohn's disease or colitis by monitoring metabolites of the 6-MP and/or thiopurine methyltransferase activity and setting dosing based on the results. U.S. Pat. Nos. 6,692,771 and 6,680,068 and U.S. Patent Application Publications 20030077306 and 20020160049 describe emulsion formulations that may help the penetration of 6-MP into the body, while U.S. Pat. Nos. 6,602,521 and 6,372,254, and U.S. Patent Application Publications 20030133976 and 20020164371 describe drug delivery systems that might improve the therapeutics of 6-MP. None of these latter patents show data demonstrating improved bioavailability or therapeutic outcomes with 6-MP. The need still exists for formulations for improved delivery of 6-MP that improve the bioavailability thereof.

Standard 6-MP tablets (described in Physician's Desk Reference 57^{th} Edition, 2003, page 1615-1618) reach full dissolution after about an hour under acidic dissolution conditions using a USP type II dissolution unit with paddles rotating at 50 rpm. 50% dissolution is reached at between 10 and 15 minutes. This rate of dissolution is not as fast as would be desirable. One method of improving the rate of dissolution of poorly soluble powders is to micronize them. In the case of 6-MP, micronization does little to improve the rate of dissolution of formulated tablets when compared to the standard formulation. The lack of improved rate of dissolution makes such tablets unlikely to show improved bioavailability when compared to the standard formulation. Further improvements to the formulation are clearly needed.

### SUMMARY OF INVENTION

The present invention relates to a pharmaceutical composition of 6-mercaptopurine wherein the 6-mercaptopurine is formulated into a dosage form and comprises an enteric coating such as EUDRAGIT® L. The enteric coating substantially prevents release of the 6-mercaptopurine in the stomach. The pharmaceutical composition may also have a delay coating which delays release of the 6-mercaptopurine for a period of time after the pharmaceutical composition has passed through the stomach. In one embodiment, the enteric coating and delay coating delay drug release from the dosage form for at least one hour after the dosage form has left the stomach.

In one embodiment, the dosage form is a tablet and the enteric coating coats the tablet. In another embodiment, the dosage form is powder, granules, or pellets in a capsule and the enteric coating coats the outside of the capsule. In another embodiment, the dosage form is pellets in a capsule, wherein the pellets are individually coated with the enteric coating.

Another aspect of the invention relates to a pharmaceutical composition of 6-mercaptopurine wherein the 6-mercaptopurine is formulated into a delayed release dosage form which releases the drug in a burst after the delay.

In another embodiment, the pharmaceutical composition of 6-mercaptopurine is formulated into a dosage form and coated with an enteric coating and, optionally, a delay coating, and is any of the pharmaceutical compositions of 6-mercaptopurine described herein (i.e., any of the spray granulated forms, from solvents or basic ethanolic water in a fluidized bed or other devices, which are described herein as giving enhanced rate of release or enhanced bioavailability as compared to the standard formulation). Alternatively, the enterically coated formulation of 6-mercaptopurine can be the standard formulation that has been provided with an enteric coating and, optionally, a delay coating.

Enterically coated compositions of the present invention include compositions of 6-mercaptopurine which, prior to coating, give improved rates of release of 6-mercaptopurine when tested in a dissolution bath. It has been found that by granulating solutions of 6-mercaptopurine and pharmaceutical carriers, and forming tablets therefrom, compositions are produced that improve the rate of dissolution of the 6-mercaptopurine. It has been further found that improvement in the rate of dissolution of the 6-mercaptopurine leads to an improvement in the bioavailability of the 6-mercaptopurine. Such compositions can be combined with an enteric coating so that the improved dissolution and improved bioavailability occur after a delay, as for example, after the compositions have been administered orally and have passed through the stomach. The enteric coating then dissolves in the intestine and the improved release characteristics are then exhibited. This produces a valuable combination of delayed release followed by improved delivery kinetics which cannot be matched by prior art formulations. In preferred embodiments, the compositions are provided with a delay coating under the enteric coating so that the release of 6-mercaptopurine is delayed for a period of time after the composition has left the stomach and moved into the intestines. In certain embodiments, the period of time can be one, two, or three hours. By the choice of proper delay coating, the delay in release of 6-mercaptopurine can be set to a desired predetermined time.

In the following descriptions of enterically coated pharmaceutical compositions and formulations, as well as in the descriptions of methods of making enterically coated pharmaceutical compositions and formulations, it should be understood that the enterically coated pharmaceutical compositions and formulations may be provided with a delay coating under the enteric coating, if so desired.

In one embodiment, the invention relates to an enterically coated pharmaceutical composition comprising 6-mercaptopurine wherein, when tested prior to coating, the pharmaceutical composition exhibits dissolution of the 6-mercaptopurine greater than 50% within seven minutes when the dissolution of a tablet comprising 50 mg of the pharmaceutical composition comprising 6-mercaptopurine is measured in 900 ml of 0.1N HCl at 37° C. in a USP type II device using paddles rotating at 50 rpm.

In another embodiment, the invention relates to an enterically coated pharmaceutical composition comprising 6-mercaptopurine wherein, when tested prior to coating, the time to reach 50% dissolution of the 6-mercaptopurine is reduced by at least about 30% compared to the standard formulation when the dissolution of a tablet comprising the pharmaceutical composition comprising 6-mercaptopurine is measured in 900 ml of 0.1N HCl at 37° C. in a USP type II device using paddles rotating at 50 rpm.

In another embodiment, the invention relates to an enterically coated pharmaceutical composition comprising 6-mercaptopurine wherein the bioavailability of the 6 mercaptopurine is improved by at least about 15% when the non-coated pharmaceutical composition is dosed to a mammal as compared to the standard formulation.

In another embodiment, the invention relates to an enterically coated pharmaceutical composition comprising 6-mercaptopurine and a potassium, sodium, magnesium, ammonium, or calcium salt of a pharmaceutically acceptable acid.

In another embodiment, the invention relates to an enterically coated pharmaceutical composition comprising 6-mercaptopurine and a potassium, sodium, magnesium, ammonium, or calcium salt of a pharmaceutically acceptable acid selected from the group consisting of acetic acid, ascorbic acid, benzoic acid, citric acid, and tartaric acid. In certain embodiments, the composition, when tested prior to coating, exhibits enhanced solubility in aqueous acid as compared to the standard formulation.

In another embodiment, the invention relates to an enterically coated pharmaceutical composition comprising 6-mercaptopurine and potassium citrate.

In another embodiment, the invention relates to an enterically coated pharmaceutical composition comprising 6-mercaptopurine wherein the 6-mercaptopurine was spray granulated from a solution onto an acceptable pharmaceutical carrier powder.

In another embodiment, the invention relates to an enterically coated pharmaceutical composition comprising 6-mercaptopurine wherein the 6-mercaptopurine was spray granulated from a solution onto an acceptable pharmaceutical carrier powder wherein the spray granulation was carried out in a fluidized bed.

In another embodiment, the invention relates to an enterically coated pharmaceutical composition comprising 6-mercaptopurine wherein the 6-mercaptopurine was spray granulated from a solution onto an acceptable pharmaceutical carrier powder wherein the solvent for the solution of 6-mercaptopurine comprises a solvent selected from the group consisting of dimethylformamide, dimethylacetamide, dimethylsulfoxide, and mixtures thereof.

In another embodiment, the invention relates to an enterically coated pharmaceutical composition comprising 6-mercaptopurine wherein the 6-mercaptopurine was spray granulated from a solution onto an acceptable pharmaceutical carrier powder wherein the solvent for the solution of 6-mercaptopurine comprises a solvent selected from the group consisting of water and an at least about stoichiometric amount of a pharmaceutically acceptable base, ethanol and an at least about stoichiometric amount of a pharmaceutically acceptable base, or ethanol/water mixtures and an at least about stoichiometric amount of a pharmaceutically acceptable base.

In another embodiment, the invention relates to an enterically coated pharmaceutical composition comprising 6-mercaptopurine wherein the 6-mercaptopurine was spray granulated from a solution onto an acceptable pharmaceutical carrier powder wherein the solvent for the solution of 6-mercaptopurine comprises a solvent selected from the group consisting of ethanol/water/potassium hydroxide, ethanol/water/sodium hydroxide, and ethanol/potassium hydroxide.

In another embodiment, the invention relates to an enterically coated pharmaceutical composition comprising 6-mercaptopurine wherein the 6-mercaptopurine was spray granulated from a solution onto an acceptable pharmaceutical carrier powder wherein the solvent for the solution of 6-mercaptopurine comprises ethanol/potassium hydroxide or ethanol/water/potassium hydroxide.

In another embodiment, the invention relates to an enterically coated pharmaceutical composition comprising 6-mercaptopurine wherein the 6-mercaptopurine was spray granulated from a solution onto an acceptable pharmaceutical carrier powder wherein the pharmaceutical carrier powder comprises a powder selected from the group consisting of lactose, starch, microcrystalline cellulose, calcium phosphate, powdered cellulose, sorbitol, and sucrose.

In another embodiment, the invention relates to an enterically coated pharmaceutical composition comprising 6-mercaptopurine wherein the 6-mercaptopurine was spray granulated from a solution onto a pharmaceutical carrier powder that comprises lactose or microcrystalline cellulose.

In another embodiment, the invention relates to an enterically coated pharmaceutical composition comprising 6-mercaptopurine wherein the 6-mercaptopurine was spray granulated from a solution onto an acceptable pharmaceutical carrier powder wherein the pharmaceutical carrier powder was pre-sprayed with a solution of a pharmaceutically acceptable acid in a molar amount that is greater than the molar amount of potassium hydroxide or other pharmaceutically acceptable base in the 6-mercaptopurine solution applied to the pharmaceutical carrier powder.

In another embodiment, the invention relates to an enterically coated pharmaceutical composition comprising 6-mercaptopurine wherein the 6-mercaptopurine was spray granulated from a solution onto an acceptable pharmaceutical carrier powder wherein the pharmaceutical carrier powder was pre-sprayed with a solution comprising an acid selected from the group consisting of acetic acid, ascorbic acid, benzoic acid, citric acid, and tartaric acid.

In another embodiment, the invention relates to an enterically coated pharmaceutical composition comprising 6-mercaptopurine wherein the 6-mercaptopurine was spray granulated from a solution onto an acceptable pharmaceutical carrier powder wherein the pharmaceutical carrier powder was pre-sprayed with a solution of citric acid.

In another embodiment, the invention relates to an enterically coated pharmaceutical composition comprising about 3% to about 20% of 6-mercaptopurine and about 4% to about 30% of potassium citrate.

In another embodiment, the invention relates to an enterically coated pharmaceutical composition comprising about 8% 6-mercaptopurine and about 5% potassium citrate.

In another embodiment, the invention relates to an enterically coated pharmaceutical composition comprising about 3% to about 20% of 6-mercaptopurine wherein the 6-mercaptopurine was spray granulated from solution onto an acceptable pharmaceutical carrier powder wherein the pharmaceutical carrier powder was pre-sprayed with a solution of citric acid.

In another aspect of the invention, the invention relates to a method of making a pharmaceutical composition of 6-mercaptopurine comprising the spray granulation of a solution of 6-mercaptopurine onto a pharmaceutical carrier, followed by enterically coating the pharmaceutical composition to provide for delayed release of the 6-mercaptopurine.

In another embodiment, the invention relates to a method of making a pharmaceutical composition of 6-mercaptopurine comprising the spray granulation of a solution of 6-mercaptopurine onto a pharmaceutical carrier wherein the 6-mercaptopurine is dissolved in a solvent that comprises a solvent selected from the group consisting of dimethylformamide, dimethylacetamide, dimethylsulfoxide, and mixtures thereof, followed by enterically coating the pharmaceutical composition to provide for delayed release of the 6-mercaptopurine.

In another embodiment, the invention relates to a method of making a pharmaceutical composition of 6-mercaptopurine comprising the spray granulation of a solution of 6-mercaptopurine onto a pharmaceutical carrier wherein the 6-mercaptopurine is dissolved in a solvent that comprises a solvent selected from the group consisting of water and an at least about stoichiometric amount of a pharmaceutically acceptable base, ethanol and an at least about stoichiometric amount of a pharmaceutically acceptable base, and ethanol/water mixtures and an at least about stoichiometric amount of a pharmaceutically acceptable base, followed by enterically coating the pharmaceutical composition to provide for delayed release of the 6-mercaptopurine.

In another embodiment, the invention relates to a method of making a pharmaceutical composition of 6-mercaptopurine comprising the spray granulation of a solution of 6-mercaptopurine onto a pharmaceutical carrier wherein the 6-mercaptopurine is dissolved in a solvent that comprises a solvent selected from the group consisting of ethanol/water/potassium hydroxide, ethanol/water/sodium hydroxide, and ethanol/potassium hydroxide, followed by enterically coating the pharmaceutical composition to provide for delayed release of the 6-mercaptopurine. In certain embodiments, the solvent consists essentially of ethanol/water/potassium hydroxide, ethanol/water/sodium hydroxide, or ethanol/potassium hydroxide.

In another embodiment, the invention relates to a method of making a pharmaceutical composition of 6-mercaptopurine comprising the spray granulation of a solution of 6-mercaptopurine onto a pharmaceutical carrier wherein the 6-mercaptopurine is dissolved in ethanol/potassium hydroxide, or ethanol/water/potassium hydroxide, followed by enterically coating the pharmaceutical composition to provide for delayed release of the 6-mercaptopurine.

In another embodiment, the invention relates to a method of making a pharmaceutical composition of 6-mercaptopurine comprising the spray granulation of a solution of 6-mercaptopurine onto a pharmaceutical carrier wherein the pharmaceutical carrier comprises a powder selected from the group consisting of lactose, starch, microcrystalline cellulose, calcium phosphate, powdered cellulose, sorbitol, and sucrose, followed by enterically coating the pharmaceutical composition to provide for delayed release of the 6-mercaptopurine.

In another embodiment, the invention relates to a method of making a pharmaceutical composition of 6-mercaptopurine comprising the spray granulation of a solution of 6-mercaptopurine onto a pharmaceutical carrier comprising lactose powder or microcrystalline cellulose, followed by enterically coating the pharmaceutical composition to provide for delayed release of the 6-mercaptopurine.

In another embodiment, the invention relates to a method of making a pharmaceutical composition of 6-mercaptopurine comprising the spray granulation of a solution of 6-mercaptopurine onto a pharmaceutical carrier wherein the pharmaceutical carrier was pre-sprayed with a solution of a pharmaceutically acceptable acid in a molar amount that is greater than the molar amount of potassium hydroxide or other pharmaceutically acceptable base in the 6-mercaptopurine solution applied to the pharmaceutical carrier, followed by enterically coating the pharmaceutical composition to provide for delayed release of the 6-mercaptopurine.

In another embodiment, the invention relates to a method of making a pharmaceutical composition of 6-mercaptopurine comprising the spray granulation of a solution of 6-mercaptopurine onto a pharmaceutical carrier using a fluidized bed granulator, followed by enterically coating the pharmaceutical composition to provide for delayed release of the 6-mercaptopurine.

In another embodiment, the invention relates to a method of spray granulating a solution of 6-mercaptopurine onto a pharmaceutical carrier to make a formulation of 6-mercaptopurine having enhanced solubility properties such that the 6-mercaptopurine dissolves in 0.1N HCl to an extent of greater than 50% within seven minutes, followed by enterically coating the formulation to provide for delayed release of the 6-mercaptopurine. In another embodiment, the invention relates to a method of spray granulating a solution of 6-mercaptopurine onto a pharmaceutical carrier to make a formulation of 6-mercaptopurine having enhanced solubility properties such that the time to reach 50% dissolution of the 6-mercaptopurine formulation is reduced by at least about 30% compared to the standard formulation when the dissolution of a tablet comprising 50 mg of 6-mercaptopurine is measured in 900 ml of 0.1N HCl at 37° C. in a USP type II device using paddles rotating at 50 rpm, followed by enterically coating the formulation to provide for delayed release of the 6-mercaptopurine.

In another embodiment, the invention relates to a method of spray granulating a solution of 6-mercaptopurine onto a pharmaceutical carrier to make a formulation of 6-mercaptopurine having enhanced solubility properties such that the 6-mercaptopurine dissolves in 0.1N HCl to an extent of greater than 50% within seven minutes, wherein the method comprises dissolving 6-mercaptopurine in a solvent that comprises a solvent selected from the group consisting of dimethylformamide, dimethylacetamide, dimethylsulfoxide, and mixtures thereof, followed by enterically coating the formulation to provide for delayed release of the 6-mercaptopurine. In certain embodiments, the solvent consists essentially of dimethylformamide, dimethylacetamide, dimethylsulfoxide, or mixtures thereof. In another embodiment, the invention relates to a method of spray granulating a solution of 6-mercaptopurine onto a pharmaceutical carrier to make a formulation of 6-mercaptopurine having enhanced solubility properties such that the time to reach 50% dissolution of the 6-mercaptopurine formulation is reduced by at least about 30% compared to the standard formulation when the dissolution of a tablet comprising 50 mg of 6 mercaptopurine is measured in 900 ml of 0.1N HCl at 37° C. in a USP type II device using paddles rotating at 50 rpm, wherein the method comprises dissolving 6-mercaptopurine in a solvent that comprises a solvent selected from the group consisting of dimethylformamide, dimethylacetamide, dimethylsulfoxide, and mixtures thereof, followed by enterically coating the formulation to provide for delayed release of the 6-mercaptopurine. In certain embodiments, the solvent consists essentially of dimethylformamide, dimethylacetamide, dimethylsulfoxide, or mixtures thereof.

In another embodiment, the invention relates to a method of spray granulating a solution of 6-mercaptopurine onto a pharmaceutical carrier to make a formulation of 6-mercaptopurine having enhanced solubility properties such that the 6-mercaptopurine dissolves in 0.1N HCl to an extent of greater than 50% within seven minutes, wherein the method comprises dissolving 6-mercaptopurine in a solvent that comprises a solvent selected from the group consisting of water and an at least about stoichiometric amount of a pharmaceutically acceptable base, ethanol and an at least about stoichiometric amount of a pharmaceutically acceptable base, and ethanol/water mixtures and an at least about stoichiometric amount of a pharmaceutically acceptable base, followed by enterically coating the formulation to provide for delayed release of the 6-mercaptopurine. In another embodiment, the invention relates to a method of spray granulating a solution of 6-mercaptopurine onto a pharmaceutical carrier to make a formulation of 6-mercaptopurine having enhanced solubility properties such that the time to reach 50% dissolution of the 6-mercaptopurine formulation is reduced by at least about 30% compared to the standard formulation when the dissolution of a tablet comprising 50 mg of 6-mercaptopurine is measured in 900 ml of 0.1N HCl at 37° C. in a USP type II device using paddles rotating at 50 rpm, wherein the method comprises dissolving 6-mercaptopurine in a solvent that comprises a solvent selected from the group consisting of water and an at least about stoichiometric amount of a pharmaceutically acceptable base, ethanol and an at least about stoichiometric amount of a pharmaceutically acceptable base, and ethanol/water mixtures and an at least about stoichiometric amount of a pharmaceutically acceptable base, followed by enterically coating the formulation to provide for delayed release of the 6-mercaptopurine. In certain embodiments, the solvent consists essentially of water and an at least about stoichiometric amount of a pharmaceutically acceptable base, ethanol and an at least about stoichiometric amount of a pharmaceutically acceptable base, or ethanol/water mixtures and an at least about stoichiometric amount of a pharmaceutically acceptable base.

In another embodiment, the invention relates to a method of spray granulating a solution of 6-mercaptopurine onto a pharmaceutical carrier to make a formulation of 6-mercaptopurine having enhanced solubility properties such that the 6-mercaptopurine dissolves in 0.1N HCl to an extent of greater than 50% within seven minutes, wherein the method comprises dissolving 6-mercaptopurine in a solvent that comprises a solvent selected from the group consisting of ethanol/water/potassium hydroxide, ethanol/water/sodium hydroxide, and ethanol/potassium hydroxide, followed by enterically coating the formulation to provide for delayed release of the 6-mercaptopurine. In another embodiment, the invention relates to a method of spray granulating a solution of 6-mercaptopurine onto a pharmaceutical carrier to make a formulation of 6-mercaptopurine having enhanced solubility properties such that the time to reach 50% dissolution of the 6-mercaptopurine formulation is reduced by at least about 30% compared to the standard formulation when the dissolution of a tablet comprising 50 mg of 6-mercaptopurine is measured in 900 ml of 0.1N HCl at 37° C. in a USP type II device using paddles rotating at 50 rpm, wherein the method comprises dissolving 6-mercaptopurine in a solvent selected from the group consisting of ethanol/water/potassium hydroxide, ethanol/water/sodium hydroxide, and ethanol/potassium hydroxide, followed by enterically coating the formulation to provide for delayed release of the 6-mercaptopurine. In certain embodiments, the solvent consists essentially of ethanol/water/potassium hydroxide, ethanol/water/sodium hydroxide, or ethanol/potassium hydroxide.

In another embodiment, the invention relates to a method of spray granulating a solution of 6-mercaptopurine onto a pharmaceutical carrier to make a formulation of 6-mercaptopurine having enhanced solubility properties such that the 6-mercaptopurine dissolves in 0.1N HCl to an extent of greater than 50% within seven minutes, wherein the method comprises dissolving 6-mercaptopurine in ethanol/potassium hydroxide or ethanol/water/potassium hydroxide, followed by enterically coating the formulation to provide for delayed release of the 6-mercaptopurine. In another embodiment, the invention relates to a method of spray granulating a solution of 6-mercaptopurine onto a pharmaceutical carrier to make a formulation of 6-mercaptopurine having enhanced solubility properties such that the time to reach 50% dissolution of the 6-mercaptopurine formulation is reduced by at least about 30% compared to the standard formulation when the dissolution of a tablet comprising 50 mg of 6-mercaptopurine is measured in 900 ml of 0.1N HCl at 37° C. in a USP type II device using paddles rotating at 50 rpm, wherein the method comprises dissolving 6-mercaptopurine in ethanol/potassium hydroxide or ethanol/water/potassium hydroxide, followed by enterically coating the formulation to provide for delayed release of the 6-mercaptopurine.

In another embodiment, the invention relates to a method of spray granulating a solution of 6-mercaptopurine onto a pharmaceutical carrier to make a formulation of 6-mercaptopurine having enhanced solubility properties such that the 6-mercaptopurine dissolves in 0.1N HCl to an extent of greater than 50% within seven minutes, wherein the pharmaceutical carrier comprises a powder selected from the group consisting of lactose, starch, microcrystalline cellulose, calcium phosphate, powdered cellulose, sorbitol and sucrose, followed by enterically coating the formulation to provide for delayed release of the 6-mercaptopurine. In another embodiment, the invention relates to a method of spray granulating a solution of 6-mercaptopurine onto a pharmaceutical carrier to make a formulation of 6-mercaptopurine having enhanced solubility properties such that the time to reach 50% dissolution of the 6-mercaptopurine formulation is reduced by at least about 30% compared to the standard formulation when the dissolution of a tablet comprising 50 mg of 6-mercaptopurine is measured in 900 ml of 0.1N HCl at 37° C. in a USP type II device using paddles rotating at 50 rpm, wherein the pharmaceutical carrier comprises a powder selected from the group consisting of lactose, starch, microcrystalline cellulose, calcium phosphate, powdered cellulose, sorbitol and sucrose, followed by enterically coating the formulation to provide for delayed release of the 6-mercaptopurine.

In another embodiment, the invention relates to a method of spray granulating a solution of 6-mercaptopurine onto a pharmaceutical carrier to make a formulation of 6-mercaptopurine having enhanced solubility properties such that the 6-mercaptopurine dissolves in 0.1N HCl to an extent of greater than 50% within seven minutes, wherein the pharmaceutical carrier comprises lactose powder, followed by enterically coating the formulation to provide for delayed release of the 6-mercaptopurine. In another embodiment, the invention relates to a method of spray granulating a solution of 6-mercaptopurine onto a pharmaceutical carrier to make a formulation of 6-mercaptopurine having enhanced solubility properties such that the time to reach 50% dissolution of the 6-mercaptopurine formulation is reduced by at least about 30% compared to the standard formulation when the dissolution of a tablet comprising 50 mg of 6-mercaptopurine is measured in 900 ml of 0.1N HCl at 37° C. in a USP type II device using paddles rotating at 50 rpm, wherein the pharmaceutical carrier comprises lactose powder or microcrystalline cellulose, followed by enterically coating the formulation to provide for delayed release of the 6-mercaptopurine.

In another embodiment, the invention relates to a method of spray granulating a solution of 6-mercaptopurine onto a pharmaceutical carrier to make a formulation of 6-mercaptopurine having enhanced solubility properties such that the 6-mercaptopurine dissolves in 0.1N HCl to an extent of greater than 50% within seven minutes, wherein the pharmaceutical carrier was pre-sprayed with a solution of a pharmaceutically acceptable acid in a molar amount that is greater than the molar amount of potassium hydroxide or other pharmaceutically acceptable base in the 6-mercaptopurine solution applied to the pharmaceutical carrier, followed by enterically coating the formulation to provide for delayed release of the 6-mercaptopurine. In another embodiment, the invention relates to a method of spray granulating a solution of 6-mercaptopurine onto a pharmaceutical carrier to make a formulation of 6-mercaptopurine having enhanced solubility properties such that the time to reach 50% dissolution of the 6-mercaptopurine formulation is reduced by at least about 30% compared to the standard formulation when the dissolution of a tablet comprising 50 mg of 6-mercaptopurine is measured in 900 ml of 0.1N HCl at 37° C. in a USP type II device using paddles rotating at 50 rpm, wherein the pharmaceutical carrier was pre-sprayed with a solution of a pharmaceutically acceptable acid in a molar amount that is greater than the molar amount of potassium hydroxide in the 6-mercaptopurine solution applied to the pharmaceutical carrier, followed by enterically coating the formulation to provide for delayed release of the 6-mercaptopurine.

In another embodiment, the invention relates to a method of spray granulating a solution of 6-mercaptopurine onto a pharmaceutical carrier to make a formulation of 6-mercaptopurine having enhanced solubility properties such that the 6-mercaptopurine dissolves in 0.1N HCl to an extent of greater than 50% within seven minutes, wherein the spray granulating uses a fluidized bed granulator, followed by enterically coating the formulation to provide for delayed release of the 6-mercaptopurine.

In another embodiment, the invention relates to a method of spray granulating a solution of 6-mercaptopurine onto a pharmaceutical carrier to make a formulation of 6-mercaptopurine having enhanced solubility properties such that the time to reach 50% dissolution of the 6-mercaptopurine formulation is reduced by at least about 30% compared to the standard formulation when the dissolution of a tablet comprising 50 mg of 6-mercaptopurine is measured in 900 ml of 0.1N HCl at 37° C. in a USP type II device using paddles rotating at 50 rpm, wherein the spray granulating uses a fluidized bed granulator, followed by enterically coating the formulation to provide for delayed release of the 6-mercaptopurine.

In another aspect of the invention, the invention relates to a method of making a pharmaceutical composition of 6-mercaptopurine having enhanced bioavailability properties such that when dosing said pharmaceutical composition to a mammal the bioavailability is improved by at least about 15%, where the method comprises enterically coating the pharmaceutical composition to provide for delayed release of the 6-mercaptopurine.

In another embodiment, the invention relates to a method of making a pharmaceutical composition of 6-mercaptopurine having enhanced bioavailability properties such that when dosing said composition to a mammal the bioavailability is improved by at least about 15%, the method comprising the spray granulation of a solution of 6-mercaptopurine onto a pharmaceutical carrier wherein the 6-mercaptopurine is dissolved in a solvent comprising a solvent selected from the group consisting of dimethylformamide, dimethylacetamide, dimethylsulfoxide, and mixtures thereof, followed by enterically coating the pharmaceutical composition to provide for delayed release of the 6-mercaptopurine. In certain embodiment, the solvent consists essentially of dimethylformamide, dimethylacetamide, dimethylsulfoxide, or mixtures thereof.

In another embodiment, the invention relates to a method of making a pharmaceutical composition of 6-mercaptopurine having enhanced bioavailability properties such that when dosing said composition to a mammal the bioavailability is improved by at least about 15%, the method comprising the spray granulation of a solution of 6-mercaptopurine onto a pharmaceutical carrier wherein the 6-mercaptopurine is dissolved in a solvent comprising a solvent selected from the group consisting of water and an at least about stoichiometric amount of a pharmaceutically acceptable base, ethanol and an at least about stoichiometric amount of a pharmaceutically acceptable base, and ethanol/water mixtures and an at least about stoichiometric amount of a pharmaceutically acceptable base, followed by enterically coating the pharmaceutical composition to provide for delayed release of the 6-mercaptopurine. In certain embodiments, the solvent consists essentially of water and an at least about stoichiometric amount of a pharmaceutically acceptable base, ethanol and an at least about stoichiometric amount of a pharmaceutically acceptable base, or ethanol/water mixtures and an at least about stoichiometric amount of a pharmaceutically acceptable base.

In another embodiment, the invention relates to a method of making a pharmaceutical composition of 6-mercaptopurine having enhanced bioavailability properties such that when dosing said composition to a mammal the bioavailability is improved by at least about 15%, the method comprising the spray granulation of a solution of 6-mercaptopurine onto a pharmaceutical carrier wherein the solution is 6-mercaptopurine dissolved in a solvent comprising a solvent selected from the group consisting of ethanol/water/potassium hydroxide, ethanol/water/sodium hydroxide, and ethanol/potassium hydroxide, followed by enterically coating the pharmaceutical composition to provide for delayed release of the 6-mercaptopurine.

In another embodiment, the invention relates to a method of making a pharmaceutical composition of 6-mercaptopurine having enhanced bioavailability properties such that when dosing said composition to a mammal the bioavailability is improved by at least about 15%, the method comprising the spray granulation of a solution of 6-mercaptopurine onto a pharmaceutical carrier wherein the solution is 6-mercaptopurine dissolved in ethanol/potassium hydroxide or ethanol/water/potassium hydroxide, followed by enterically coating the pharmaceutical composition to provide for delayed release of the 6-mercaptopurine.

In another embodiment, the invention relates to a method of making a pharmaceutical composition of 6-mercaptopurine having enhanced bioavailability properties such that when dosing said composition to a mammal the bioavailability is improved by at least about 15%, the method comprising the spray granulation of a solution of 6-mercaptopurine onto a pharmaceutical carrier wherein the pharmaceutical carrier comprises a powder selected from the group consisting of lactose, starch, microcrystalline cellulose, calcium phosphate, powdered cellulose, sorbitol, and sucrose, followed by enterically coating the pharmaceutical composition to provide for delayed release of the 6-mercaptopurine.

In another embodiment, the invention relates to a method of making a pharmaceutical composition of 6-mercaptopurine having enhanced bioavailability properties such that when dosing said composition to a mammal the bioavailability is improved by at least about 15%, comprising the spray granulation of a solution of 6-mercaptopurine onto a pharmaceutical carrier comprising lactose, followed by enterically coating the pharmaceutical composition to provide for delayed release of the 6-mercaptopurine.

In another embodiment, the invention relates to a method of making a pharmaceutical composition of 6-mercaptopurine having enhanced bioavailability properties such that when dosing said composition to a mammal the bioavailability is improved by at least about 15%, the method comprising the spray granulation of a solution of 6-mercaptopurine onto a pharmaceutical carrier wherein the pharmaceutical carrier was pre-sprayed with a solution of a pharmaceutically acceptable acid in a molar amount that is greater than the molar amount of potassium hydroxide or other pharmaceutically acceptable base in the 6-mercaptopurine solution applied to the pharmaceutical carrier, followed by enterically coating the pharmaceutical composition to provide for delayed release of the 6-mercaptopurine.

In another embodiment, the invention relates to a method of making a pharmaceutical composition comprising 6-mercaptopurine having enhanced bioavailability properties such that when dosing said composition to a mammal the bioavailability is improved by at least about 15% compared to the standard formulation, the method comprising the spray granulation of a solution of 6-mercaptopurine onto a pharmaceutical carrier using a fluidized bed granulator, followed by enterically coating the pharmaceutical composition to provide for delayed release of the 6-mercaptopurine.

Another aspect of this invention is a method of dosing to a patient suffering from Crohn's disease, arthritis, or colitis a pharmaceutical composition comprising 6-mercaptopurine in a delayed release formulation.

In one embodiment of this method, the release of 6-mercaptopurine is delayed by at least one hour after the dosage form has left the stomach

In another aspect, the invention relates to a method of dosing an enterically coated pharmaceutical composition comprising 6-mercaptopurine to patients in need of said drug wherein, when tested prior to coating, the composition displays enhanced solubility in aqueous acid compared to the standard formulation.

In another embodiment, the invention relates to a method of dosing an enterically coated pharmaceutical composition comprising 6-mercaptopurine to patients in need of said drug wherein, when tested prior to coating, the composition displays enhanced solubility in aqueous acid such that the 6-mercaptopurine dissolves in 0.1N HCl to an extent of greater than 50% within seven minutes or wherein the time to reach 50% dissolution of the 6-mercaptopurine is reduced by at least about 30% compared to the standard formulation when the dissolution of a tablet comprising 50 mg of 6-mercaptopurine is measured in 900 ml of 0.1N HCl at 37° C. in a USP type II device using paddles rotating at 50 rpm.

In another embodiment, the invention relates to a method of dosing an enterically coated pharmaceutical composition comprising 6-mercaptopurine to patients in need of said drug wherein the bioavailability is improved by at least about 15% when dosing to a mammal as compared to the standard formulation.

In another embodiment, the invention relates to a method of dosing an enterically coated pharmaceutical composition comprising 6-mercaptopurine to patients in need of said drug to treat leukemia or other cancers wherein, when tested prior to coating, the composition displays enhanced solubility in aqueous acid as compared to the standard formulation.

In another embodiment, the invention relates to a method of dosing an enterically coated pharmaceutical composition comprising 6-mercaptopurine to patients in need of said drug to treat Crohn's disease, arthritis, or colitis wherein, when tested prior to coating, the composition displays enhanced solubility in aqueous acid as compared to the standard formulation.

In another embodiment, the invention relates to a method of dosing an enterically coated pharmaceutical composition comprising 6-mercaptopurine to patients in need of said drug to treat leukemia or other cancers wherein the bioavailability of the 6-mercaptopurine is improved by at least about 15% when dosing the non-coated pharmaceutical composition to a mammal as compared to the standard formulation.

In another embodiment, the invention relates to a method of dosing an enterically coated pharmaceutical composition comprising 6-mercaptopurine to patients in need of said drug to treat Crohn's disease, arthritis, or colitis wherein the bioavailability is improved by at least about 15% when dosing the non-coated pharmaceutical composition to a mammal as compared to the standard formulation.

In another embodiment, the invention relates to a method of dosing an enterically coated pharmaceutical composition comprising 6-mercaptopurine to patients in need of said drug to treat leukemia or other cancers wherein the dose administered is reduced by at least about 15% and achieves the same bioavailability as the standard formulation.

In another embodiment, the invention relates to a method of dosing an enterically coated pharmaceutical composition comprising 6-mercaptopurine to patients in need of said drug to treat Crohn's disease, arthritis, or colitis wherein the dose administered is reduced by at least about 15% and achieves the same bioavailability as the standard formulation.

Another aspect of this invention is a method of treating a patient with Crohn's disease, arthritis, or colitis with a delayed release formulation of 6-mercaptopurine.

In one embodiment of this invention, the release of 6-mercaptopurine is delayed by at least one hour after the dosage form has left the stomach.

In another embodiment of this invention, the release of 6-mercaptopurine is delayed by at least one hour after the dosage form has left the stomach and the release is over a short period of time thereafter (burst release).

### DESCRIPTION OF THE DRAWINGS

FIG. 1 shows the dissolution of an uncoated 6-mercaptopurine composition (6-MP-113) versus PURINETHOL® in 0.1 N HCl (see Example 1).

FIG. 2 shows the dissolution of an uncoated 6-mercaptopurine composition (6-MP-113 batch) vs. PURINETHOL® in 0.1N HCl (see Example 2).

FIG. 3 shows the average pharmacokinetic profile of 6-mercaptopurine for an uncoated pharmaceutical composition (6-MP-IB batch) vs. the standard formulation (PURINETHOL®) (see Example 4).

FIG. 4 shows the dissolution of uncoated 6-mercaptopurine tablets prepared as in Example 3. -▴-=PURINETHOL®; -♦-=tablets prepared with microcrystalline cellulose; -▪-=tablets prepared with lactose; -x-=lactose tablets, 70% ethanol, 30% water, n=3 (average of three tablets).

FIG. 5 shows the time delay in dissolution provided by a coating of 120 mg (-♦-) or 180 mg (-▪-).

### DESCRIPTION OF THE PREFERRED EMBODIMENTS

The present invention is directed to enterically coated compositions of 6-mercaptopurine that exhibit a delay in release of 6-mercaptopurine. The delayed release provides for high local concentrations of of 6-mercaptopurine in the intestine.

In certain embodiments, the present invention is directed to enterically coated compositions of 6-mercaptopurine wherein the non-coated compositions provide an improved rate of dissolution when tested in a dissolution bath and show improved bioavailability characteristics when dosed to mammals.

As used herein, the “standard formulation” is the formulation described in the Physician's Desk Reference, 57^{th} edition, 2003, pages 1615-1618 and sold in the United States under the brand name PURINETHOL®.

As used herein, the term “enhanced solubility properties” or “enhanced solubility” of a material or composition of the present invention means an improved rate of dissolution of the material or composition of the present invention or an improved extent of dissolution of the material or composition of the present invention as compared to the standard formulation.

As used herein, the term “improved bioavailability” refers to the increase in concentration of a drug in the body fluid provided by the compositions of the present invention as compared to the concentration of the drug in the body fluid from the standard formulation under identical conditions. Drug bioavailability is proportional to, and is typically measured by, the total area under the curve (AUC) of the concentration of the drug found in blood or plasma versus time when measured in a pharmacokinetic trial in a human or an animal. The AUC may be expressed as AUCt, i.e. the area under the curve to the last measured time point, or AUC_{I}, i.e. the area under the curve extrapolated to infinite time. The improvement in bioavailability is measured by the percent increase in the average AUC of the subjects in the trial when dosing the improved formulation as compared to the average AUC of the same subjects obtained by dosing of the standard formulation of the drug. Alternatively, the AUC ratio of the test formulation (AUCf) to the AUC of the reference formulation (AUCr) may be calculated on a per subject basis and then averaged. A percent of the average ratio (AUCf/AUCr) above 100% is then the improvement in bioavailability.

As used herein, the term “slight stoichiometric excess” refers to a stoichiometric excess of about 0.1% to about 30%, preferably about 0.5% to about 15%, more preferably about 1% to about 5%, in terms of mole percent.

As used herein, “pre-sprayed” refers to spraying the pharmaceutical carrier powder with the acid before the acid-sprayed pharmaceutical carrier is contacted with the solution of 6-mercaptopurine.

As used herein, “powder” in reference to a pharmaceutical carrier refers to particles of the pharmaceutical carrier having a size range of 1 to 800 micron, more preferably 2 to 500 microns, and most preferably 2 to 100 microns or 50 to 400 microns, depending on the material.

As used herein, “6-MP” refers to 6-mercaptopurine.

As used herein, “substantially no release of 6-mercaptopurine occurs before passage of the composition through the stomach” means that no more than about 10%, preferably no more than about 5%, and even more preferably no more than about 1% of the 6-mercaptopurine in a composition is released before passage of the composition through the stomach. In other words, at least about 90%, preferably at least about 95%, and even more preferably at least about 99% of the 6-mercaptopurine in the composition is released after the composition has passed through the stomach.

As used herein, “substantially no release of 6-mercaptopurine occurs until at least about a predetermined period of time after passage of the composition through the stomach” means that no more than about 10%, preferably no more than about 5%, and even more preferably no more than about 1% of the 6-mercaptopurine in a composition is released before the predetermined period of time after passage of the composition through the stomach. In other words, at least about 90%, preferably at least about 95%, and even more preferably at least about 99% of the 6-mercaptopurine in the composition is released after the predetermined period of time after the composition has passed through the stomach.

Enteric coatings are coatings applied to tablets, capsules, pellets, or granules that have the property of being insoluble in acid and impermeable to acid but soluble and/or permeable at or about neutral pH. Examples of such coatings are the methacrylic acid copolymers NF which are fully polymerized copolymers of methacrylic acid and an acrylic or methacrylic ester and are known as EUDRAGIT® L and EUDRAGIT® S, as well as cellulose acetate phthalate. In a preferred embodiment, EUDRAGIT® L is used. The enteric coat can be applied as a water dispersion or as a solution in organic solvents, with ethanol or isopropanol being the preferred organic solvents. Typically, enteric coatings are applied with plasticizers in the solution so that the film will contain the plasticizer and maintain a certain degree of flexibility. Typical plasticizers are polyethylene glycols (PEG) and triethyl citrate. In an embodiment of this invention, the plasticizer content can range from 5% to 50%, more preferably 10% to 30%, based on the weight of the polymer. The formulations optionally contain talc to help prevent the tablets from sticking together during processing. In one most preferred embodiment, the solution applied to tablets of 6-MP contained about 6% EUDRAGIT® L, about 0.6% triethyl citrate, about 3% talc, about 5% water and about 85.4% isopropanol.

In one embodiment, the dosage form to be coated is in the form of tablets. When coating tablets, a coating of 20 to 30 mg is usually sufficient to prevent drug release in the stomach and allow for facile drug release at the start of the small intestine. In one embodiment of this invention, it is desired to delay the release of the drug for at least an hour after the tablet has left the stomach and started traveling down the small intestine. In one embodiment, the dosage form is coated with a delay coating under the enteric coating. The enteric coating prevents drug release in the stomach while the delay coating is triggered upon entry into the small intestine and imparts the delay desired. In a preferred embodiment, the delay coating is another layer, or a thicker layer, of the enteric coating itself. In one preferred embodiment, the weight of the enteric coating applied to the tablets was about 100 mg. This weight of coating gave no release of drug in simulated gastric solution and gave a delay in drug release of 75 to 90 minutes when tested in simulated small intestinal buffer of pH 6.8. Tablets coated with about 120 mg similarly gave a delay of about 120 minutes while tablets coated with about 180 mg gave a delay of 180 minutes when tested in simulated intestinal buffer. Any dosage form containing 6-mercaptopurine could be coated with enteric coatings. In another embodiment, the dosage form is powder, granules or pellets which are filled in a capsule and the enteric coating coats the outside of the capsule. One skilled in the art could readily determine the amount of coating to be applied to the outside of the capsule to achieve the desired delay. In another embodiment, the dosage form is pellets in a capsule wherein the pellets themselves are coated with the enteric coating. The most preferred form is the enteric coated tablet as described with about 120 mg of enteric coating containing 10% plasticizer giving an about 2 hour delay of drug release after leaving the stomach.

In one embodiment of this invention, the pharmaceutical composition of 6-mercaptopurine that is enterically coated is the standard 6-mercaptopurine formulation as appears in the PDR. A more preferred embodiment of a 6-mercaptopurine dosage form comprising an enteric coating is any of the pharmaceutical compositions of 6-mercaptopurine described herein (i.e., any of the spray granulated forms, from solvents or basic ethanolic water in a fluidized bed or other device which are described herein as giving enhanced rate of release or enhanced bioavailability). A most preferred embodiment comprises a EUDRAGIT® L coating comprising 10% triethyl citrate on a formulation comprising 30 to 50 mg 6-mercaptopurine, spray granulated in a fluidized bed dryer from a basic ethanol-water solution that optionally contains a binder such as PVP, where the composition was spray granulated onto lactose or microcrystalline cellulose that was pre-loaded with a slight stoichiometric excess of citric acid and further formulated and processed into tablets. The coating level is most preferably about 120 mg per tablet.

One embodiment of the invention is directed to enterically coated formulations of 6-MP that provide for a delayed release of 6-MP that comprise 6-MP formulated into granulates by first dissolving the 6-MP in an organic solvent. Examples of solvents that can be used to dissolve the 6-MP to an extent sufficient to be able to apply the solution to a pharmaceutical powder for further processing are dimethylformamide, dimethylacetamide, and dimethylsulfoxide, or mixtures thereof. Lactose, starch, microcrystalline cellulose, calcium phosphate, powdered cellulose, sorbitol or sucrose are examples of pharmaceutically acceptable powders that can be used as powders for this granulation. Other pharmaceutical excipient powders are known in the art and may also be used. In a more preferred embodiment, the organic solvent solution of 6-MP is spray granulated on to the powder so as to form a uniform coating. A preferred method of performing this spray granulation is by using a fluidized bed granulator. A more preferred embodiment uses lactose as the pharmaceutical powder upon which the 6-MP is granulated, and a yet more preferred embodiment uses dimethylformamide to form the granulation solution. In a more preferred embodiment of the invention a lactose granulate is formed that comprises, on a weight/weight (w/w) basis, 1 to 35% 6-MP, more preferably 5 to 20% 6-MP, and most preferably about 13% 6-MP. These granulates are then mixed with other tablet excipients and formed into tablets comprising 0.5 mg to 150 mg of 6-MP for an approximate tablet weight of 500 mg with an about 50 mg dose the most preferred. Alternatively, the dose of 6-MP can be controlled by changing tablet weight using any of the preferred, more preferred, or most preferred granulates.

Tablets that comprise these formulations of 6-MP have improved dissolution properties. When testing these tablets prior to enteric coating in 900 ml of 0.1N HCl at 37° C. in a USP apparatus II dissolution tester with paddles rotating at 50 rpm, the rate of dissolution is greatly enhanced compared to the standard formulation. The time to 50% of dissolution is below seven minutes more preferably below five minutes and exhibits a more than 30% reduction in the time to 50% dissolution, more preferably a more than 50% reduction in time to 50% dissolution, when compared to the standard formulation.

A more preferred embodiment of this invention is directed to enterically coated 6-MP formulations that comprise 6-MP formulated into granulates by first dissolving the 6-MP in ethanol containing at least about a stoichiometric amount of base, water containing at least about a stoichiometric amount of base, or mixtures of ethanol/water containing at least about a stoichiometric amount of base. The base may be selected from any pharmaceutically acceptable base such as the hydroxide or carbonate salts of potassium, sodium, magnesium, ammonium, or calcium, with potassium hydroxide being preferred. Optionally, a binder such as polyvinylpyrrolidone (PVP) may be added to the solution. This basic solution of 6-MP is granulated onto a pharmaceutical carrier selected from the group of lactose, starch, microcrystalline cellulose, calcium phosphate, powdered cellulose, sorbitol, or sucrose. Other pharmaceutical excipient powders are known in the art and may also be used. In a preferred embodiment, the basic solvent solution of 6-MP is spray granulated on to the powder so as to form a uniform coating. A preferred method of performing this spray granulation is by using a fluidized bed granulator. A more preferred embodiment uses lactose as the pharmaceutical powder upon which the 6-MP is granulated and a most preferred embodiment uses an ethanol/water solvent mixture and potassium hydroxide as the base. In another more preferred embodiment, microcrystalline cellulose is used as the pharmaceutical powder upon which the 6-MP is granulated, and a most preferred embodiment uses an ethanol/water solvent mixture and potassium hydroxide as the base. The basic granulate is neutralized with a slight stoichiometric excess of any pharmaceutically acceptable acid. Examples of such acids are acetic acid, ascorbic acid, benzoic acid, citric acid, and tartaric acid. In a more preferred embodiment the acid selected is citric acid. In a more preferred embodiment, the pharmaceutically acceptable acid is precoated in a slight stoichiometric excess onto the pharmaceutically acceptable carrier before it is used in the granulation with the basic organic solution of 6-MP. In a more preferred embodiment, the pharmaceutically acceptable carrier is lactose and the pharmaceutically acceptable acid that is preloaded in a slight stoichiometric excess is citric acid. A more preferred mode for applying the acid is spray granulation and a most preferred method uses a fluidized bed granulator. In a preferred embodiment of the invention a lactose granulate is formed that comprises 1 to 35% 6-MP, more preferably 5 to 20% 6-MP, and most preferably about 11% 6-MP. In another preferred embodiment of the invention, a microcrystalline cellulose granulate is formed that comprises 1 to 35% 6-MP, more preferably 5 to 20% 6-MP, and most preferably about 11% 6-MP. These granulates further comprise salts of pharmaceutically acceptable acids, more preferably the sodium or potassium salts of acetic acid, ascorbic acid, benzoic acid, citric acid, or tartaric acid and most preferably the potassium salt of citric acid. The potassium citrate is present in about a stoichiometric amount compared to the 6-MP. These granulates are then mixed with other tablet excipients and formed into tablets comprising 0.5 mg to 150 mg of 6-MP for an approximate total tablet weight of 650 mg with an about 50 mg dose of 6-MP being the most preferred. Alternatively, the dose of 6-MP can be controlled by changing tablet weight using any of the preferred, more preferred, or most preferred granulates. In another embodiment, the final dosage form comprises about 3% to about 20% of 6-mercaptopurine and about 2% to about 30% of potassium citrate and more preferably about 5% to about 15% of 6-MP and about 2% to about 20% potassium citrate, and most preferably about 8% 6-mercaptopurine and about 5% potassium citrate.

Tablets that comprise these enterically coated formulations of 6-MP have improved dissolution properties, following the delay imparted by the enteric coating and the delay coating, if present. When testing these tablets (in uncoated form) in 900 ml of 0.1N HCl at 37° C. in a USP apparatus II dissolution tester with paddles rotating at 50 rpm, the rate of dissolution is greatly enhanced as compared to the standard formulation. The time to 50% of dissolution is below seven minutes, more preferably below five minutes, and exhibits a more than 30% reduction in the time to 50% dissolution, more preferably a more than 50% reduction in time to 50% dissolution, when compared to the standard formulation.

Standard formulation 6-MP tablets reach full dissolution after about an hour under acidic dissolution conditions using a USP type II dissolution unit with paddles rotating at 50 rpm. 50% dissolution is reached at between 10 and 15 minutes. Improved rates of dissolution are defined herein as a time to 50% dissolution of less than or equal to about seven minutes, more preferably less than or equal to about five minutes, or a more than 30% reduction in the time to 50% dissolution, more preferably a more than or equal to 50% reduction in the time to 50% dissolution, compared to the standard formulation.

One aspect of the present invention is a method of forming enterically coated 6-MP formulations that comprises granulating 6-MP into granulates by first dissolving the 6-MP in an organic solvent. Examples of solvents that can be used to dissolve the 6-MP to an extent sufficient to be able to apply the solution to a pharmaceutical powder for further processing are dimethylformamide, dimethylacetamide, and dimethylsulfoxide, or mixtures thereof. Lactose, starch, microcrystalline cellulose, calcium phosphate, powdered cellulose, sorbitol, or sucrose are examples of pharmaceutically acceptable powders that can be used as powders for this granulation. Other pharmaceutical excipient powders are known in the art and may also be used. In a more preferred embodiment the organic solvent solution of 6-MP is spray granulated on to the powder so as to form a uniform coating. A preferred method of performing this spray granulation is by using a fluidized bed granulator. A more preferred embodiment uses lactose as the pharmaceutical powder upon which the 6-MP is granulated and a yet more preferred embodiment uses dimethylformamide to form the granulation solution. In a more preferred embodiment of the invention, a lactose granulate is formed that comprises 1 to 35% 6-MP, more preferably 5 to 20% 6-MP, and most preferably about 13% 6-MP. These granulates are then mixed with other tablet excipients and formed into tablets comprising 0.5 mg to 150 mg of 6-MP for an approximate tablet weight of 500 mg with an about 50 mg dose being the most preferred. Alternatively, the dose of 6-MP can be controlled by changing tablet weight using any of the preferred, more preferred, or most preferred granulates. After formation of the granulates as described above, the granulates are provided with an enteric coating, and, optionally, a delay coating.

Tablets that comprise formulations of 6-MP made by this method have improved dissolution properties. When testing these tablets in uncoated form in 900 ml of 0.1N HCl at 37° C. in a USP apparatus II dissolution tester with paddles rotating at 50 rpm, the rate of dissolution is greatly enhanced compared to the standard formulation. The time to 50% of dissolution is below seven minutes, more preferably below five minutes, and exhibits a more than 30% reduction in the time to 50% dissolution, more preferably a more than 50% reduction in time to 50% dissolution, when compared to the standard formulation.

A more preferred embodiment of this invention is a method of making enterically coated 6-MP formulations that comprises granulating 6-MP into granulates by first dissolving the 6-MP in ethanol containing at least a stoichiometric amount of base, water containing at least a stoichiometric amount of base, or mixtures of ethanol/water containing at least a stoichiometric amount of base. The base may be selected from any pharmaceutically acceptable base such as the hydroxide or carbonate salts of potassium, sodium, magnesium, ammonium, or calcium, with potassium hydroxide being more preferred. Optionally, a binder such as polyvinylpyrrolidone (PVP) may be added to the solution. This basic solution of 6-MP is granulated onto a pharmaceutical carrier selected from the group consisting of lactose, starch, microcrystalline cellulose, calcium phosphate, powdered cellulose, sorbitol, and sucrose. Other pharmaceutical excipient powders are known in the art and may also be used. The granulates so formed are then provided with an enteric coating, and, optionally a delay coating.

In a more preferred embodiment the basic solvent solution of 6-MP is spray granulated on to the powder so as to form a uniform coating. A preferred method of performing this spray granulation is by using a fluidized bed granulator. A more preferred embodiment uses lactose as the pharmaceutical powder upon which the 6-MP is granulated, and a most preferred embodiment uses an ethanol/water solvent mixture and potassium hydroxide as the base. In another more preferred embodiment, microcrystalline cellulose is used as the pharmaceutical powder upon which the 6-MP is granulated, and a most preferred embodiment uses an ethanol/water solvent mixture and potassium hydroxide as the base. The basic granulate is neutralized with a stoichiometric excess of any pharmaceutically acceptable acid. Examples of such acids are acetic acid, ascorbic acid, benzoic acid, citric acid, and tartaric acid. In a more preferred embodiment, the acid is citric acid. In a more preferred embodiment, the pharmaceutically acceptable acid is preloaded in a slight stoichiometric excess onto the pharmaceutically acceptable carrier before it is used in the granulation with the basic organic solution of 6-MP. In a more preferred embodiment, the pharmaceutically acceptable carrier is lactose and the pharmaceutically acceptable acid that is preloaded in a slight stoichiometric excess is citric acid. A more preferred method for applying the acid is spray granulation, and a most preferred method uses a fluidized bed granulator. In a preferred embodiment of the invention, a lactose granulate is formed that comprises 1 to 35% 6-MP, preferably 5 to 20% 6-MP, and most preferably about 11% 6-MP. In another preferred embodiment of the invention, a microcrystalline cellulose granulate is formed that comprises 1 to 35% 6-MP, more preferably 5 to 20% 6-MP, and most preferably about 11% 6-MP. These granulates further comprise salts of pharmaceutically acceptable acids, preferably the sodium or potassium salts of acetic acid, ascorbic acid, benzoic acid, citric acid, or tartaric acid, and most preferably the potassium salt of citric acid. The potassium citrate is present in about a stoichiometric amount compared to the 6-MP. These granulates are then mixed with other tablet excipients and formed into tablets comprising 0.5 mg to 150 mg of 6-MP for an approximate total tablet weight of 650 mg, with an about 50 mg dose of 6-MP in the tablet being most preferred. Alternatively, the dose of 6-MP can be controlled by changing tablet weight using any of the preferred, more preferred, or most preferred granulates. In another embodiment, the final dosage form comprises about 3% to about 20% of 6-mercaptopurine and about 2% to about 30% of potassium citrate, preferably about 5% to about 15% of 6-MP and about 2% to about 20% potassium citrate, and most preferably about 8% 6-mercaptopurine and about 5% potassium citrate. The tablets formed as described above are then provided with an enteric coating, and, optionally, a delay coating.

Tablets that comprise enterically coated formulations of 6-MP made by this method have improved dissolution properties after the delay imparted by the enteric coating and the optional delay coating. When testing these tablets in their uncoated form in 900 ml of 0.1N HCl at 37° C. in a USP apparatus II dissolution tester with paddles rotating at 50 rpm, the rate of dissolution is greatly enhanced compared to the standard formulation. The time to 50% of dissolution is below seven minutes, preferably below five minutes, and exhibits a more than 30% reduction in the time to 50% dissolution, preferably a more than 50% reduction in time to 50% dissolution, when compared to the standard formulation.

Another aspect of the invention is a method of producing enterically coated compositions of 6-mercaptopurine which provide enhanced bioavailability compared to the standard formulation. The enhanced bioavailability may be a rise in average AUCt or AUC_{I} of about 5% or more, preferably a rise of about 15% or more, and most preferably a rise of 20% or more. Alternatively, the average ratio of the individual AUCt values for the test and reference formulations is about 1.05 or more, preferably 1.15 or more, and most preferably 1.20 or more. One embodiment of this aspect of the invention is a method of making enterically coated 6-MP formulations that comprises granulating 6-MP into granulates by first dissolving the 6-MP in an organic solvent. Examples of solvents that can be used to dissolve the 6-MP to an extent sufficient to be able to apply the solution to a pharmaceutical powder for further processing are dimethylformamide, dimethylacetamide, and dimethylsulfoxide, or mixtures thereof. Lactose, starch, microcrystalline cellulose, calcium phosphate, powdered cellulose, sorbitol, or sucrose are examples of pharmaceutically acceptable powders that can be used as powders for this granulation. Other pharmaceutical excipient powders are known in the art and may also be used. In a more preferred embodiment, the organic solvent solution of 6-MP is spray granulated on to the powder so as to form a uniform coating. A preferred method of performing this spray granulation is by using a fluidized bed granulator. A more preferred embodiment uses lactose as the pharmaceutical powder upon which the 6-MP is granulated, and a yet more preferred embodiment uses dimethylformamide to form the granulation solution. In a more preferred embodiment of the invention, a lactose granulate is formed that comprises 1 to 35% 6-MP, preferably 5 to 20% 6-MP, and most preferably about 13% 6-MP. These granulates are then mixed with other tablet excipients and formed into tablets comprising 0.5 mg to 150 mg of 6-MP for an approximate total tablet weight of 500 mg, with an about 50 mg of 6-MP in that tablet being the dose most preferred. Alternatively, the dose of 6-MP can be controlled by changing tablet weight using any of the preferred, more preferred, or most preferred granulates. The tablets formed as described above are then provided with an enteric coating, and, optionally, a delay coating.

A more preferred embodiment of this invention is a method of producing enterically coated 6-MP formulations that comprises granulating 6-MP into granulates by first dissolving the 6-MP in ethanol containing at least about a stoichiometric amount of base, water containing at least about a stoichiometric amount of base, or mixtures of ethanol/water containing at least about a stoichiometric amount of base. The base may be selected from any pharmaceutically acceptable base such as the hydroxide or carbonate salts of potassium, sodium, magnesium, ammonium, or calcium, with potassium hydroxide being more preferred. Optionally, a binder such as polyvinylpyrrolidone (PVP) may be added to the solution. This basic solution of 6-MP is granulated onto a pharmaceutical carrier selected from the group of lactose, starch, microcrystalline cellulose, calcium phosphate, powdered cellulose, sorbitol, and sucrose. Other pharmaceutical excipient powders are known in the art and may also be used. In a more preferred embodiment the basic solvent solution of 6-MP is spray granulated on to the powder so as to form a uniform coating. A preferred method of performing this spray granulation is by using a fluidized bed granulator. A more preferred embodiment uses lactose as the pharmaceutical powder upon which the 6-MP is granulated, and a most preferred embodiment uses an ethanol/water solvent mixture and potassium hydroxide as the base. In another more preferred embodiment, microcrystalline cellulose is used as the pharmaceutical powder upon which the 6-MP is granulated, and a most preferred embodiment uses an ethanol/water solvent mixture and potassium hydroxide as the base. The basic granulate is neutralized with a slight stoichiometric excess of any pharmaceutically acceptable acid. Examples of such acids are acetic acid, ascorbic acid, benzoic acid, citric acid, and tartaric acid. In a more preferred embodiment, the acid selected is citric acid. In a more preferred embodiment, the pharmaceutically acceptable acid is preloaded in a slight stoichiometric excess onto the pharmaceutically acceptable carrier before it is used in the granulation with the basic organic solution of 6-MP. In a more preferred embodiment, the pharmaceutically acceptable carrier is lactose and the pharmaceutically acceptable acid that is preloaded in an about slight stoichiometric excess is citric acid. A more preferred mode for applying the acid is spray granulation and a most preferred method uses a fluidized bed granulator. In a preferred embodiment of the invention a lactose granulate is formed that comprises 1 to 35% 6-MP, preferably 5 to 20% 6-MP, and most preferably about 11% 6-MP. In another preferred embodiment of the invention, a microcrystalline cellulose granulate is formed that comprises 1 to 35% 6-MP, more preferably 5 to 20% 6-MP, and most preferably about 11% 6-MP. These granulates further comprise salts of pharmaceutically acceptable acids, preferably the sodium or potassium salts of acetic acid, ascorbic acid, benzoic acid, citric acid, or tartaric acid, and most preferably the potassium salt of citric acid. The potassium citrate is present in about a stoichiometric amount compared to the 6-MP. These granulates are then mixed with other tablet excipients and formed into tablets comprising 0.5 mg to 150 mg of 6-MP for an approximate total tablet weight of 650 mg, with an about 50 mg dose of 6-MP in the tablet being preferred. Alternatively, the dose of 6-MP can be controlled by changing tablet weight using any of the preferred, more preferred, or most preferred granulates. In another embodiment the final dosage form comprises about 3% to about 20% of 6 mercaptopurine and about 2% to about 30% of potassium citrate, preferably about 5% to about 15% of 6-MP and about 2% to about 20% potassium citrate, and most preferably about 8% 6-mercaptopurine and about 5% potassium citrate. The tablets formed as described above are then provided with an enteric coating, and, optionally, a delay coating.

Tablets that comprise formulations of 6-MP made by this method have improved dissolution properties and improved bioavailability following the delay in release imparted by the enteric coating and the optional delay coating. The dissolution properties and bioavailability of the non-coated tablets are improved by more than 5%, preferably by more than 15%, and most preferably by more than 20%, when tested in beagle dogs.

In one embodiment, the present invention provides a pharmaceutical dosage form comprising:
a core comprising 6-mercaptopurine; and
an enteric coating;
wherein the enteric coating imparts a delay in the release of the 6-mercaptopurine following oral administration of the dosage form such that release of 6-mercaptopurine occurs after passage of the dosage form through the stomach.

In certain embodiments, the dosage form comprises a delay coating which imparts a further delay in the release of the 6-mercaptopurine such that substantially no release of 6-mercaptopurine occurs until a predetermined period of time after passage of the dosage form through the stomach. In certain embodiments, the predetermined period of time is at least about one hour, at least about two hours, or at least about three hours.

In certain embodiments, substantially no release of 6-mercaptopurine occurs before passage of the dosage form through the stomach.

In certain embodiments, the core comprises:
(a) 6-mercaptopurine and a potassium, sodium, magnesium, ammonium, or calcium salt of a pharmaceutically acceptable acid; or
(b) a uniform coating of 6-mercaptopurine over a pharmaceutical carrier powder.

In certain embodiments, the core comprises:
(a) 6-mercaptopurine and a potassium, sodium, magnesium, ammonium, or calcium salt of a pharmaceutically acceptable acid; and
(b) a uniform coating of 6-mercaptopurine over a pharmaceutical carrier powder.

In certain embodiments, the core comprises a pharmaceutically acceptable acid selected from the group consisting of acetic acid, ascorbic acid, benzoic acid, citric acid, and tartaric acid. In certain embodiments, the core comprises potassium citrate.

In certain embodiments, the 6-mercaptopurine is spray granulated from a solution onto a pharmaceutical carrier powder to form a uniform coating of 6-mercaptopurine over the pharmaceutical carrier powder. The spray granulation may be carried out in a fluidized bed. The solution of 6-mercaptopurine may comprise:
(a) a solvent selected from the group consisting of dimethylformamide, dimethylacetamide, dimethylsulfoxide, and mixtures thereof; or
(b) a solvent selected from the group consisting of: water and an at least about stoichiometric amount of a pharmaceutically acceptable base, ethanol and an at least about stoichiometric amount of a pharmaceutically acceptable base, and ethanol/water mixtures and an at least about stoichiometric amount of a pharmaceutically acceptable base.

In certain embodiments, the solvent comprises ethanol/water/potassium hydroxide, ethanol/water/sodium hydroxide, or ethanol/potassium hydroxide. In such embodiments, the pharmaceutical carrier powder may be pre-sprayed with a solution of a pharmaceutically acceptable acid in a molar amount that is greater than the molar amount of the potassium hydroxide or sodium hydroxide in the 6-mercaptopurine solution applied to the pharmaceutical carrier powder.

In certain embodiments, the pharmaceutical carrier powder comprises a powder selected from the group consisting of: lactose, starch, microcrystalline cellulose, calcium phosphate, powdered cellulose, sorbitol, and sucrose.

In certain embodiments, the core has the following characteristics prior to enteric coating:
(a) the dissolution rate of the 6-mercaptopurine is greater than 50% within seven minutes when measured in 900 ml of 0.1N HCl at 37° C. in a USP type II device using paddles rotating at 50 rpm;
(b) the time to reach 50% dissolution of the 6-mercaptopurine is reduced by at least about 30% compared to the standard formulation when measured in 900 ml of 0.1N HCl at 37° C. in a USP type II device using paddles rotating at 50 rpm; or
c) the bioavailability of the 6-mercaptopurine is improved by at least about 15% when the core is dosed to a mammal as compared to the standard formulation.

In certain embodiments, the dissolution of a tablet comprising the core is measured in part (a) or part (b) above. In certain embodiments, the tablet comprises 50 mg of 6-mercaptopurine.

In certain embodiments, when measured prior to enteric coating, the dissolution of the 6-mercaptopurine in the core is greater than 50% within five minutes when measured in 900 ml of 0.1N HCl at 37° C. in a USP type II device using paddles rotating at 50 rpm.

In certain embodiments, the improved bioavailability is a rise in average AUCt or AUC_{I} of about 5%, or about 15%, or about 20%. In other embodiments, the improved bioavailability is a rise in the average ratio of the individual AUCt values, as compared to the standard formulation, of about 1.05, or about 1.15, or about 1.20.

In certain embodiments, the core comprises about 3% to about 20% of 6-mercaptopurine and about 2% to about 30% of potassium citrate and the core exhibits enhanced solubility in aqueous acid as compared to the standard formulation. In certain embodiments, the core comprises about 8% 6-mercaptopurine and about 5% potassium citrate.

The present invention provides a method of dosing 6-mercaptopurine to patients in need of treatment with 6-mercaptopurine comprising administering a pharmaceutical dosage form comprising:
a core comprising 6-mercaptopurine; and
an enteric coating;
   wherein the 6-mercaptopurine is released after a delay of at least one hour after the dosage form leaves the stomach.

The present invention provides a method of treating leukemia or other cancers, Crohn's disease, arthritis, or ulcertative colitis comprising administering a pharmaceutical dosage form comprising:
a core comprising 6-mercaptopurine; and
an enteric coating;
   to a patient having or suspected of having leukemia or another cancer, Crohn's disease, arthritis, or ulcerative colitis wherein the 6-mercaptopurine is released after a delay of at least one hour after the dosage form leaves the stomach.

The enteric coating which coats the core may be copolymers of methacrylic acid and an acrylic or methacrylic ester such as EUDRAGIT® L or EUDRAGIT® S or may be cellulose acetate phthalate.

Another aspect of this invention is a method of treating patients in need of treatment with 6-MP by dosing them with enterically coated formulations of 6-MP. Following the delay in release of the 6-MP imparted by the enteric coating and the optional delay coating, these formulations provide for high local concentrations of of 6-mercaptopurine in the intestine. In certain embodiments, these formulations have enhanced bioavailability compared to the standard formulation. Examples of patients in need of treatment with 6-MP are patients suffering from any disease in which a cytotoxic drug is beneficial such as leukemia, especially acute lymphocytic leukemia, or other cancers, as well as patients suffering from any disease for which an immunosuppressant drug is beneficial, such as Crohn's disease, ulcerative colitis, or arthritis.

The enhanced bioavailability may be a rise in average AUCt or AUC_{I} of about 5% or more, preferably a rise of about 15% or more, and most preferably a rise of about 20% or more. Alternatively, the average ratio of the individual AUCt values for the test and reference formulations is about 1.05 or more, preferably 1.15 or more, and most preferably about 1.20 or more. One embodiment of this aspect of the invention is a method of dosing, to a mammal, 6-MP formulations that comprise granulates that were produced by first dissolving the 6-MP in an organic solvent. Examples of solvents that can be used to dissolve the 6-MP to an extent sufficient to be able to apply the solution to a pharmaceutical powder for further processing are dimethylformamide, dimethylacetamide, and dimethylsulfoxide, or mixtures thereof. Lactose, starch, microcrystalline cellulose, calcium phosphate, powdered cellulose, sorbitol, or sucrose are examples of pharmaceutically acceptable powders that can be used as powders for this granulation. Other pharmaceutical excipient powders are known in the art and may also be used. In a more preferred embodiment the organic solvent solution of 6-MP is spray granulated on to the powder so as to form a uniform coating. A preferred method of performing this spray granulation is by using a fluidized bed granulator. A more preferred embodiment uses lactose as the pharmaceutical powder upon which the 6-MP is granulated and a yet more preferred embodiment uses dimethylformamide to form the granulation solution. In a more preferred embodiment of the invention a lactose granulate is formed that comprises 1 to 35% 6-MP, more preferably 5 to 20% 6-MP and most preferably about 13% 6-MP. These granulates are then mixed with other tablet excipients and formed into tablets comprising 0.5 mg to 150 mg of 6-MP for an approximate tablet weight of 500 mg with an about 50 mg dose the most preferred. Alternatively, the dose of 6-MP can be controlled by changing tablet weight using any of the preferred, more preferred, or most preferred granulates. The tablets formed as described above are then provided with an enteric coating, and, optionally, a delay coating.

Other tablet excipients that may be used to formulate tablets comprising the pharmaceutical compositions include binders, diluents, disintegrants, lubricants, colorants, and taste masking agents. Suitable binders include microcrystalline cellulose, modified celluloses, and povidone. Suitable diluents include calcium hydrogen phosphate (CaHPO₄), anhydrous; lactose; and mannitol. Suitable disintegrants include sodium starch glycollate (type A), sodium starch glycollate (type B), and crospovidone. Suitable lubricants include sodium stearyl fumarate, dimeticone, macrogol 6000, hydrogenated castor oil, and stearic acid.

A more preferred embodiment of this invention is a method of dosing, to a mammal, enterically coated 6-MP formulations that comprise granulates that were produced by first dissolving the 6-MP in ethanol containing at least about a stoichiometric amount of base, water containing at least about a stoichiometric amount of base, or mixtures of ethanol/water containing at least about a stoichiometric amount of base. The base may be selected from any pharmaceutically acceptable base such as the hydroxide or carbonate salts of potassium, sodium, magnesium, ammonium, or calcium, with potassium hydroxide being preferred. Optionally, a binder such as polyvinylpyrrolidone (PVP) may be added to the solution. This basic solution of 6-MP is granulated onto a pharmaceutical carrier selected from the group of lactose, starch, microcrystalline cellulose, calcium phosphate, powdered cellulose, sorbitol and sucrose. Other pharmaceutical excipient powders are known in the art and may also be used. In a more preferred embodiment the basic solvent solution of 6-MP is spray granulated on to the powder so as to form a uniform coating. A preferred method of performing this spray granulation is by using a fluidized bed granulator. A more preferred embodiment uses lactose as the pharmaceutical powder upon which the 6-MP is granulated and a most preferred embodiment uses an ethanol/water solvent mixture and potassium hydroxide as the base. In another more preferred embodiment, microcrystalline cellulose is used as the pharmaceutical powder upon which the 6-MP is granulated, and a most preferred embodiment uses an ethanol/water solvent mixture and potassium hydroxide as the base. The basic granulate is neutralized with an about slight stoichiometric excess of any pharmaceutically acceptable acid. Examples of such acids are acetic acid, ascorbic acid, benzoic acid, citric acid, and tartaric acid. In a more preferred embodiment, the acid selected is citric acid. In a more preferred embodiment, the pharmaceutically acceptable acid is preloaded in a slight stoichiometric excess onto the pharmaceutically acceptable carrier before it is used in the granulation with the basic organic solution of 6-MP. In a more preferred embodiment the pharmaceutically acceptable carrier is lactose and the pharmaceutically acceptable acid that is preloaded in a slight stoichiometric excess is citric acid. A more preferred mode for applying the acid is spray granulation and a most preferred method uses a fluidized bed granulator. In a preferred embodiment of the invention, a lactose granulate is formed that comprises 1 to 35% 6-MP, more preferably 5 to 20% 6-MP, and most preferably about 11% 6-MP. In another preferred embodiment of the invention, a microcrystalline cellulose granulate is formed that comprises 1 to 35% 6-MP, more preferably 5 to 20% 6-MP, and most preferably about 11% 6-MP. These granulates further comprise salts of pharmaceutically acceptable acids, more preferably the sodium or potassium salts of acetic acid, ascorbic acid, benzoic acid, citric acid, or tartaric acid and most preferably the potassium salt of citric acid. The potassium citrate is present in about a stoichiometric amount compared to the 6-MP. These granulates are then mixed with other tablet excipients and formed into tablets comprising 0.5 mg to 150 mg of 6-MP for an approximate tablet weight of 650 mg, with an about 50 mg dose the most preferred. Alternatively, the dose of 6-MP can be controlled by changing tablet weight using any of the preferred, more preferred, or most preferred granulates. In another embodiment, the final dosage form comprises about 3% to about 20% of 6-mercaptopurine and about 2% to about 30% of potassium citrate and more preferably about 5% to about 15% of 6-MP and about 2% to about 20% potassium citrate, and most preferably about 8% 6-mercaptopurine and about 5% potassium citrate. The tablets formed as described above are then provided with an enteric coating, and, optionally, a delay coating.

In one embodiment, the patients in need of said treatment are treated with a dose similar to the dose given with the standard formulation, thereby achieving enhanced efficacy. In another embodiment, the dose of treatment is lowered so as to have the same bioavailability as the standard treatment but achieved with a lower dose of drug. The result of the treatment is the same efficacy as the standard formulation with less exposure to potent drugs and an improved side effect profile.

Another aspect of this invention is the method of treating a patient with Crohn's disease or colitis with a delayed release formulation of 6-mercaptopurine. 6-mercaptopurine or its prodrug azathioprine is typically used in the maintenance of remission in Crohn's disease. For induction of remission, these drugs in their current formulations are often inappropriate since they work slowly, typically taking more than 3 months to show an effect. They are believed to work as immunosuppressants, by suppressing the proliferation of immune cells thought to be responsible for the lesions in Crohn's disease. Both azathioprine and 6-MP are currently dosed systemically. While systemic dosing treats the entire organism, the concentration of drug at the local site of lesion in the intestines is small. By treating with a delayed release formulation and subsequently releasing the drug in a soluble form at relatively high concentration in the intestines, one can obtain greatly improved efficacy by high concentration treatment of the local foci of the immune system. This local delivery may allow 6-MP to be used for the induction of remission as well as being a better drug for maintenance of remission.

In one embodiment of this invention, the patient is treated with a dosage form in which the release of 6-mercaptopurine is delayed by at least one hour after the dosage form has left the stomach. A facile way of achieving this goal is by using an enteric coating on the dosage form that prevents drug release in the stomach and also having a delay coating under the enteric coating. In a more preferred embodiment, the delay coating is another layer, or a thicker layer, of the enteric coating. In a preferred embodiment, the drug is released after the at least one hour delay in a burst fashion, giving high local concentrations of the drug. In one embodiment, the dose of 6-mercaptopurine is 10 to 100 mg, more preferably 25 to 50 mg, and most preferably about 35 to 40 mg. In a most preferred embodiment, the dosage form with an at least one hour delay of drug delivery after leaving the stomach is any of the forms spray granulated in a fluidized bed or other device, from solvents or basic ethanolic water, which are described herein as giving enhanced rate of release or enhanced bioavailability.

Another aspect of this invention is a method of dosing to a patient suffering from Crohn's disease or colitis a pharmaceutical composition comprising 6-mercaptopurine in a delayed release formulation. A facile way of achieving this goal is by using an enteric coating on the dosage form that prevents drug release in the stomach and also having a delay coating under the enteric coating. In a more preferred embodiment, the delay coating is another layer, or a thicker layer, of the enteric coating. In a preferred embodiment, the drug is released after the at least one hour delay in a burst fashion giving high local concentrations of the drug. In one embodiment, the dose of 6-mercaptopurine is 10 to 100 mg, more preferably 25 to 50 mg, and most preferably about 35 to 40 mg. In a most preferred embodiment, the dosage form with an at least one hour delay of drug delivery after leaving the stomach is any of the forms spray granulated in a fluidized bed or other device, from solvents or basic ethanolic water, which are described herein as giving enhanced rate of release or enhanced bioavailability.

Methods of making 6-mercaptopurine are known in the art. For example, 6-mercaptopurine can be made according to the processes described in G. H. Hitchings, G. B. Elion, U.S. Pat. No. 2,697,702 or G. B. Elion, et al., J. Am. Chem. Soc. 74,411 (1952).

### EXAMPLES

### Example 1

Mercaptopurine Spray Granulated from Dimethylformamide Solution

6-Mercaptopurine (6-MP, Orion-Fermion, 13.2 gm) was dissolved in dimethylformamide (DMF, Merck, 1.25 liter) with stirring over a period of 30 minutes. Lactose (DMV, 85 gm) was charged into a fluidized bed drier/granulator (FBD) and suspended by airflow. The air inlet temperature was 70° C. The DMF solution of 6-MP was sprayed into the suspended fluidized bed at a rate that maintained a bed temperature of 36° C. Total spraying time was 6 hours. The granulated lactose was subsequently dried in the FBD at 70° C. for one hour and sieved through a 1.0 mm screen. The dry granulate (100 gm which contained 13.2 gm 6-MP) was mixed with potato starch (AVEBE, 25.9 grams), microcrystalline cellulose (Avicel 101,_FMC, 13.2 grams) and croscarmellose sodium (Ac-Di-Sol,_FMC, 3.7 grams) for 8 minutes. Magnesium stearate (Brenntag, 0.5 grams) was added and the powder mixed for a further minute. The powder was pressed into tablets using a Korsch 106 rotary tablet press, using 12 mm flat faced round punches with the inscription φβ571. Final tablet weight was 542 mg and the 6-MP content was 50 mg (6-MP-IB batch 131-016-1).

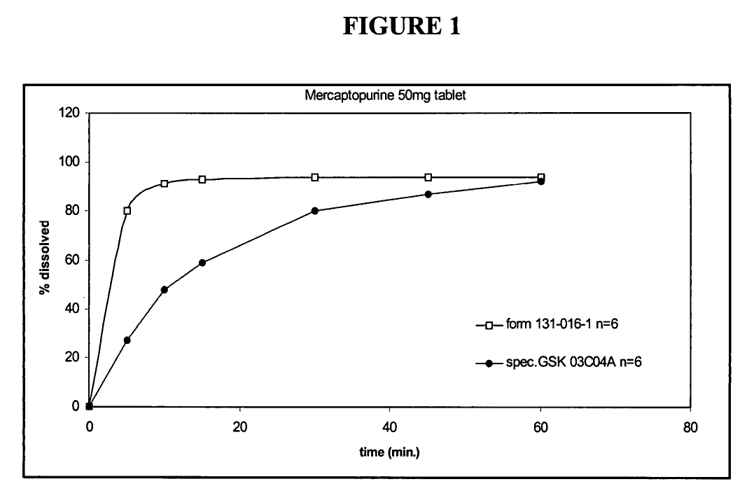

Dissolution analysis was carried out in a USP type II dissolution bath (VanKel) using 900 ml of 0.1N HCl kept at 37° C. and stirred at 50 rpm. Samples were taken at 5, 10 15, 30, 45, and 60 minutes. PURINETHOL® (batch GSK03C04A) was tested under identical conditions. The 6-MP content of the samples was measured by WV spectroscopy at 325 nm against a standard curve. The results of the measurements are given in Table 1 and shown graphically in FIG. 1.

TABLE 1
Dissolution of 6-mercaptopurine from 6-MP-IB 131-016-1 vs.
PURINETHOL ® in 0.1N HCl

| 6-MP-IB | PURINETHOL ® |
|---|---|
| 131-016-1 | GSK03C04A |

| Time | | Time | |
|---|---|---|---|
| (min) | Cumulative % | (min) | Cumulative % |
| 0 | 0 | 0 | 0 |
| 5 | 80 | 5 | 27 |
| 10 | 91 | 10 | 48 |
| 15 | 93 | 15 | 59 |
| 30 | 94 | 30 | 80 |
| 45 | 94 | 45 | 87 |
| 60 | 94 | 60 | 92 |

The results of the dissolution show that the DMF spray granulated 6-MP tablets give a much faster dissolution in 0.1N HCl than the standard formulation tablets. The time to 50% dissolution was better than halved with 80% being dissolved in 5 minutes and 91% at 10 minutes. The improved speed of dissolution of the product is expected to lead to improved bioavailability in vivo.

### Example 2

Mercaptopurine Spray Granulated from Ethanol/Water/KOH Solution

Citric acid (Merck, 4.6 gm) was dissolved in 69 ml ethanol/water (70:30). This solution was sprayed onto a bed of lactose (DMV, 80 grams) suspended in an FBD granulator using the following conditions: inlet air temperature 55° C., bed temperature 28° C. 6-mercaptopurine (Orion-Fermion, 11.4 gm) was dissolved in 430 ml ethanol/water (80:20) containing pre-dissolved potassium hydroxide (Merck, 4.0 gram). The 6-MP solution was then sprayed onto the lactose/citric acid bed in the FBD using the following conditions: inlet air temperature 55° C., bed temperature 28° C. The bed was dried in situ at 55° C. for 30 minutes. The dried granulate was passed through a 1.6 mm sieve. The dried and sieved granulate (100 grams) was mixed with potato starch (AVEBE, 26 grams), microcrystalline cellulose (Avicel 101, FMC, 11.4 grams), crospovidone (ISP Global Tech, 7.5 grams), and colloidal silicon dioxide (Degussa, 0.5 grams) for 8 minutes. Magnesium stearate (Brenntag, 2.2 gram) was added and the powder mixed for a further 2 minutes. The powder was pressed into tablets using a Korsch 106 rotary tablet press using 12 mm flat faced round punches with the inscription φβ571. Final tablet weight was 647 mg and the 6-MP content was 50 mg (6-MP-IB batch 131-018-6)

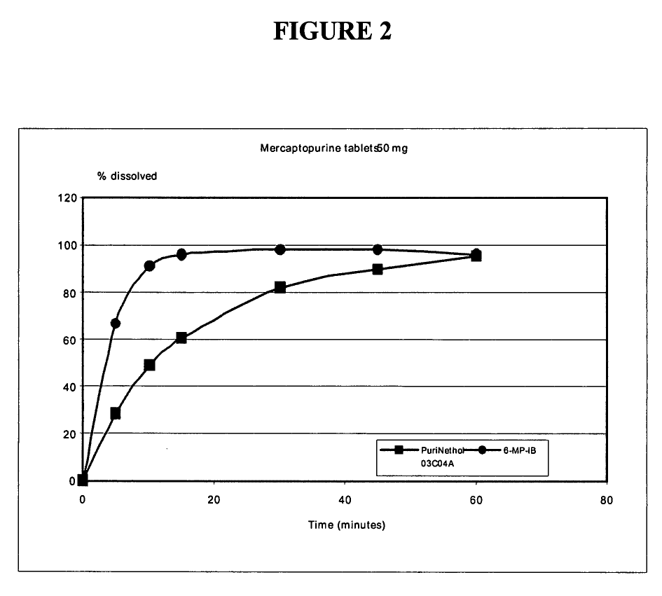

Dissolution analysis was carried out in a USP type II dissolution bath (VanKel) using 900 ml of 0.1N HCl kept at 37° C. and stirred at 50 rpm. Samples were taken at 5, 10, 15, 30, 45, and 60 minutes. PURINETHOL® (batch GSK03CD4A) was tested under identical conditions. The 6-MP content of the samples was measured by UV spectroscopy at 325 nm against a standard curve. The results of the measurements are given in Table 2 and shown graphically in FIG. 2.

TABLE 2
Dissolution of 6-mercaptopurine from 6-MP-IB 131-018-6 vs.
PURINETHOL ® in 0.1N HCl

| 6-MP-IB | PURINETHOL ® |
|---|---|
| 131-018-6 | GSK03C04A |

| Time | | Time | |
|---|---|---|---|
| (min) | Cumulative % | (min) | Cumulative % |
| 0 | 0 | 0 | 0 |
| 5 | 67 | 5 | 27 |
| 10 | 91 | 10 | 48 |
| 15 | 96 | 15 | 59 |
| 30 | 98 | 30 | 80 |
| 45 | 98 | 45 | 87 |
| 60 | 96 | 60 | 92 |

The results of the dissolution show that the basic ethanolic-water spray granulated 6-MP tablets give a much faster dissolution in 0.1N HCl than the standard formulation tablets. The time to 50% dissolution was better than halved with 67% being dissolved in 5 minutes and better than 90% at 10 minutes. The improved speed of dissolution of the product is expected to lead to improved bioavailability in vivo.

### Example 3

Tablets of 6-MP Coated on Microcrystalline Cellulose or Lactose

This example present data from tablets in which 6-MP is coated on either microcrystalline cellulose or lactose. Table 3 shows a batch formula for tablets having 40 mg of 6-MP per tablet (the batch is for ˜1000 tablets), tablet weight 523 mg using 50% ethanol by volume (44.4% by weight) in both spraying steps.

TABLE 3

| | Raw material | (g) | (g) |
|---|---|---|---|
| 1 | Lactose monohydrate | 280 | — |
| 2 | Microcrystalline Cellulose | — | 280 |
| 3 | Citric Acid anhydrate | 19.5 | 19.5 |
| 4 | Alcohol denatured or USP | 96^{#} | 96^{#} |
| 5 | Purified Water | 120 | 120 |
| 6 | Mercaptopurine | 40.0 | 40.0 |
| 7 | Potassium hydroxide | 16.2 | 16.2 |
| 8 | PVP K30 | — | 10.4 |
| 9 | Alcohol denatured or USP | 600^{#} | 600^{#} |
| 10 | Purified Water | 750 | 750 |
| 11 | Colloidal Silicon Dioxide | 1.6 | 1.6 |
| 12 | Potato Starch | 24.4 | 24.4 |
| 13 | Crospovidone | 26.4 | 26.4 |
| 14 | Microcrystalline Cellulose | 91.6 | 91.6 |
| 15 | PVP K30 | 15.6 | 5.2 |
| 16 | Magnesium Stearate | 8.0 | 8.0 |

#Density 0.8 g/mL

Manufacturing Method
Solution A.

Mix alcohol (denatured or USP) (4) with purified water (5), add and dissolve citric acid (3).

Coating Step I (Aeromatic Strea 1)

Spray solution A on to lactose monohydrate (1) or microcrystalline cellulose (MCC) (2).

Process Parameters:

| | |
|---|---|
| Atomizing air: | 1 bar |
| Nozzle: | 1.0 mm |
| Inlet temperature: | 55° C. |
| Exhaust temperature: | approx. 24° C. |
| Spray rate: | approx. 9-10 g/min |
| Airflow rate: | approx. 54 m³/h |

Solution B.

Mix alcohol (denatured or USP) (9) with purified water (10), add and dissolve potassium hydroxide (7). Add and dissolve 6-mercaptopurine (6). Optionally, PVP K30 (8) may be dissolved in this solution (either with lactose or with MCC-shown here with MCC).

Coating Step II (Aeromatic Strea 1)

Spray solution B onto the lactose monohydrate with citric acid or MCC with citric acid of coating step I.

Process Parameters:

| | |
|---|---|
| Atomizing air: | 1 bar |
| Nozzle: | 1.0 mm |
| Inlet temperature: | 55° C. |
| Exhaust temperature: | approx. 24° C. |
| Spray rate: | approx. 10-11 g/min |
| Airflow rate: | approx. 54-80 m³/h |

Drying

Dry the lactose/citric acid/potassium hydroxide/6-mercaptopurine mixture or the MCC/citric acid/potassium hydroxide/PVP/6-mercaptopurine mixture.

Process Parameters:

| | |
|---|---|
| Inlet temperature: | 55° C. |
| Exhaust temperature: | approx. 34° C. |
| Airflow rate: | approx. 54-80 m³/h |

Sieving I

Pass the lactose/citric acid/potassium hydroxide/6-mercaptopurine mixture or the MCC/citric acid/potassium hydroxide/PVP/6-mercaptopurine mixture through a 1.0 mm sieve.

Pass colloidal silicon dioxide (11) through a 1.0 mm sieve.

Mixing I

Blend the lactose/citric acid/potassium hydroxide/6-mercaptopurine mixture or the MCC/citric acid/potassium hydroxide/PVP/6-mercaptopurine mixture with colloidal silicon dioxide for 2 minutes in a cubic tumbler.

Sieving II

Pass potato starch (12), crospovidone (13), microcrystalline cellulose (14) and PVP K30 (15) through 1.0 mm sieve.

Mixing II

Blend the lactose/citric acid/potassium hydroxide/6-mercaptopurine/colloidal silicon dioxide mixture or the MCC/citric acid/potassium hydroxide/PVP/6-mercaptopurine/colloidal silicon dioxide mixture with potato starch, crospovidone, microcrystalline cellulose and PVP K30 for 8 minutes in a cubic tumbler.

Sieving III

Pass magnesium stearate (16) through a 1.0 mm sieve.

Mixing III

Blend the mixture of Mixing step H with magnesium stearate for 2 minutes in a cubic tumbler.

Tabletting

Compress the final mixture into tablets with tablet weight 523 mg (12 mm, round convex R=9.5). Resistance to crushing of 5-7 Kp, friability max. 1.0%, disintegration time <5 min.

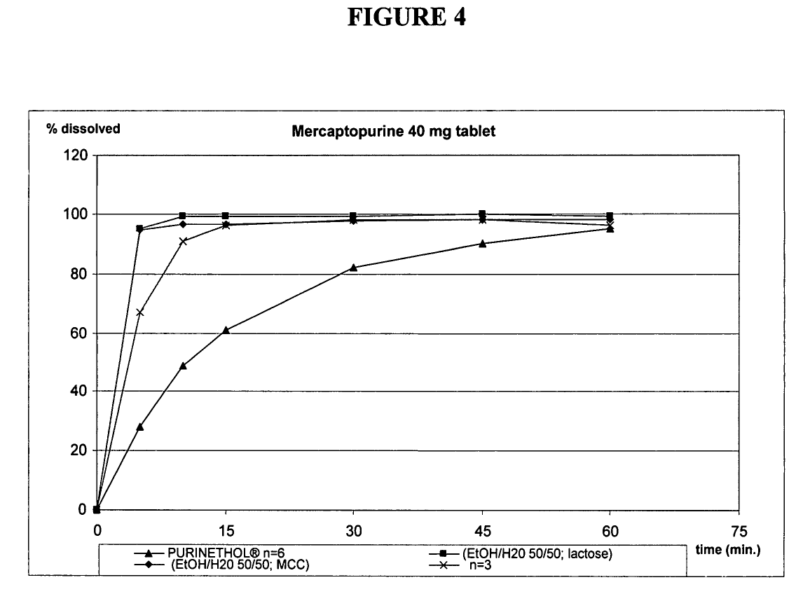

The results of the dissolution of 6-MP tablets prepared as in this example in 900 ml 0.1 N HCl at 37° C. and 50 rpm is shown in FIG. 4.

### Example 4

### A Comparative Bioavailability Study of a New Oral Formulation of 6-Mercaptopurine (6-MP-IB) vs. PURINETHOL® in Beagle Dogs

Study Objective—To determine the pharmacokinetic profile (AUCt and AUC₁, Cmax, Tmax, and half life of 6-mercaptopurine in the plasma following oral ingestion of each formulation to show improved bioavailability for 6-MP-IB

Study Design—Single center, single dose, non-randomized, open label (blinded to analyst), two treatment, two period crossover comparative bioavailability study.

Subjects—Six female beagle dogs, 2-3 years old, 9-11 kg body weight.

Study Administrations

1) PURINETHOL® (GSK): Half of a 50 mg tablet (i.e. 25 mg) of 6-mercaptopurine, Lot #A067350.
2) 6-MP-IB batch 131-018-6: Half of a 50 mg tablet (i.e. 25 mg) of 6-mercaptopurine.

The dogs received the half tablets in the fasted state (twelve hours fast). The tablets were placed in the back of the dog's throat. About 10 ml of water was squirted into the mouth with a syringe to facilitate swallowing. The mouth was examined to ensure that the tablet was swallowed.

Blood Collection and Handling

Blood samples were taken from an indwelling catheter inserted in the jugular vein at 0 hour and at 0.25, 0.5, 1.0, 1.5, 2.0, 3.0, 4.0, 5.0, and 6.0 hours post dosing. Seven milliliters of blood was collected at each time point. The blood was chilled in ice immediately after collection. Within two minutes of collection the blood was transferred to tubes containing EDTA. The blood was processed to obtain the plasma within one hour. The plasma was stabilized with dithiothreitol and frozen to −80° C.

Analyses

The analysis of 6-MP in the plasma was carried out at Anapharm Laboratories by a validated LC/MS/MS method.

Study Duration

Two study sessions with a wash out of two weeks between study sessions.

Results

The results of the analysis of 6-MP in the plasma for all the dogs are given in Table 4A for the reference PURINETHOL® and in Table 4B for the test formulation 6-MP-IB.

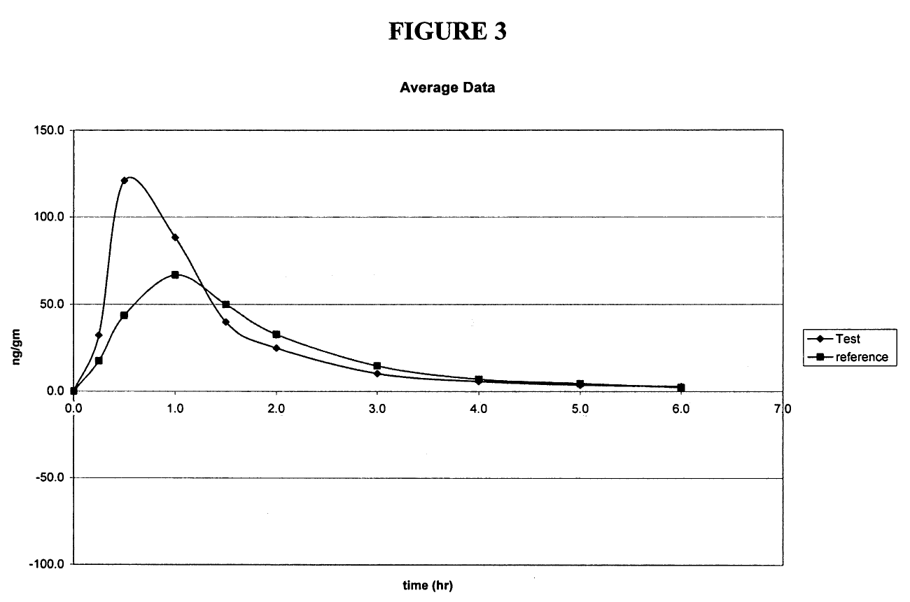

The results of the calculated pharmacokinetic parameters from the concentration data are collected in Table 5 while the results of a per dog ratio analysis are given in Table 6. The average pharmacokinetic profiles for all six dogs for each treatment are given in FIG. 3.

One can see in Table 5 that the average AUCt and AUC_{I} are both about 20% higher for the test formulation (i.e., the composition of the present invention) when compared to the standard formulation. The Cmax is almost 70% higher. In the ratio analysis, shown in Table 6, where each dog is its own control, there is an average ratio of 1.26 or a 26% rise in the bioavailability of the test versus the reference product.

FIG. 3 shows that the advantage of the faster dissolving formulation in bioavailability is in the early time points with higher drug concentrations being found shortly after drug ingestion. The Tmax for the averaged data is shorter for the test compared to reference despite the fact that the average Tmax (averaged over the individual dogs) is the same for the two formulations.

### CONCLUSIONS

The formulation provided by the present invention has been shown to give a more than 20% increase in bioavailability of 6-mercaptopurine in vivo when compared to an equivalent dose of the standard formulation. The improved bioavailability is expected to allow improved therapeutic outcomes.

TABLE 4a
6-mercaptopurine standard formulation (PURINETHOL ®) concentrations
(ng/ml)

| Subject | Period | Draw Times (Hour) | | | | | | | | | |
|---|---|---|---|---|---|---|---|---|---|---|---|
| # | # | 0.000 | 0.250 | 0.500 | 1.00 | 1.50 | 2.00 | 3.00 | 4.00 | 5.00 | 6.00 |
| 02 | 1 | <2.00 | 35.15 | 38.98 | 149.72 | 131.27 | 80.36 | 26.90 | 11.01 | 7.87 | 5.37 |
| 03 | 1 | <2.00 | <2.00 | 53.24 | 41.64 | 31.96 | 39.83 | 19.10 | 8.85 | 4.76 | 2.73 |
| 04 | 1 | <2.00 | 21.69 | 112.90 | 54.94 | 26.45 | 15.24 | 9.75 | 12.12 | 8.24 | <2.00 |
| 05 | 1 | <2.00 | 20.97 | <2.00 | 123.11 | 75.23 | 62.88 | 41.19 | 13.16 | 8.96 | 4.87 |
| 06 | 1 | <2.00 | 61.09 | 143.83 | 106.22 | 42.88 | 22.53 | 8.98 | 5.84 | 3.23 | 2.19 |
| 11 | 1 | <2.00 | <2.00 | <2.00 | 59.72 | 91.79 | 39.99 | 10.20 | 4.53 | 2.46 | 2.03 |

TABLE 4b
6-mercaptopurine (6-MP-IB 131-018-6) concentrations (ng/ml)

| Subject | Period | Draw Times (Hour) | | | | | | | | | |
|---|---|---|---|---|---|---|---|---|---|---|---|
| # | # | 0.000 | 0.250 | 0.500 | 1.00 | 1.50 | 2.00 | 3.00 | 4.00 | 5.00 | 6.00 |
| 02 | 2 | <2.00 | 25.07 | 109.97 | 181.60 | 77.10 | 37.32 | 15.22 | 8.52 | 5.29 | 3.83 |
| 03 | 2 | <2.00 | 129.92 | 159.49 | 79.27 | 77.05 | 37.12 | 11.66 | 6.64 | 3.62 | <2.00 |
| 04 | 2 | <2.00 | 30.68 | 173.75 | 99.24 | 35.45 | 21.17 | 8.88 | 4.35 | 2.71 | 8.29 |
| 05 | 2 | <2.00 | <2.00 | 380.69 | 172.31 | 59.78 | 27.99 | 20.85 | 12.50 | 8.26 | 5.91 |
| 06 | 2 | <2.00 | <2.00 | 4.61 | 104.99 | 44.09 | 53.45 | 19.34 | 10.30 | 6.69 | 4.05 |
| 11 | 2 | <2.00 | 70.75 | 139.59 | 69.21 | 24.87 | 21.03 | 5.47 | 3.15 | 2.14 | <2.00 |

TABLE 5
Pharmokinetic results of dog study of 6-Mercaptopurine

| Dog-session- | | AUCi | t½ | | Cmax |
|---|---|---|---|---|---|
| treatment | AUCt (h * ng/g) | (h * ng/g) | (h) | Tmax (h) | (ng/g) |
| 02-2-test | 235.8 | 241.7 | 1.1 | 1.0 | 181.6 |
| 03-2-test | 220.2 | 220.2 | 0.9 | 0.5 | 159.5 |
| 04-2-test | 176.1 | 188.2 | 1.0 | 0.5 | 173.8 |
| 05-2-test | 324.4 | 338.5 | 1.7 | 1.0 | 380.7 |
| 06-2-test | 154.7 | 160.6 | 1.0 | 1.0 | 105.0 |
| 11-2-test | 143.6 | 143.6 | 0.9 | 0.5 | 139.6 |
| 02-1-ref | 272.6 | 279.5 | 0.9 | 1.0 | 149.7 |
| 03-1-ref | 120.7 | 124.5 | 1.0 | 0.5 | 53.2 |
| 04-1-ref | 130.0 | 130.0 | 1.7 | 0.5 | 112.9 |
| 05-1-ref | 217.3 | 224.3 | 1.0 | 1.0 | 123.1 |
| 06-1-ref | 179.8 | 183.3 | 1.1 | 0.5 | 143.8 |
| 11-1-ref | 124.0 | 126.2 | 0.8 | 1.5 | 91.8 |
| AVG(test) | 209.1 | 215.5 | 1.1 | 0.8 | 190.0 |
| AVG (ref) | 174.1 | 178.0 | 1.1 | 0.8 | 112.4 |

TABLE 6
Ratio Analysis

| | Cmaxtest/ | AUCt-test/ |
|---|---|---|
| Dog | Cmaxref | AUCt-ref |
| 02 | 1.21 | 0.86 |
| 03 | 3.00 | 1.82 |
| 04 | 1.54 | 1.35 |
| 05 | 3.09 | 1.49 |
| 06 | 0.73 | 0.86 |
| 11 | 1.52 | 1.16 |
| AVG | 1.848 | 1.259 |

### Example 5

Enteric Coating and Delayed Release

Lactose based tablets were coated with approx. 100 mg of EUDRAGIT® L100 (core weight 526.5 mg, coated tablet weight 625.1 mg).

Composition of Coating:

TABLE 7

| Amount | Excipients |
|---|---|
| 60 g | EUDRAGIT ® L100 |
| 6 g | Triethyl citrate |
| 50 g | Water |
| 854 g | 2-Propanol |
| 30 g | Talc |
| 1000 g | Total amount |

Results of Dissolution Tests

Coated tablets were tested for 120 minutes in gastric fluid. The tablets showed no evidence of disintegration, cracking, softening, or drug release.

The tablets were tested for their dissolution rate in intestinal fluid (potassium phosphate buffer, pH 6.8). The results (n=3) are show in the table below.

TABLE 8

| Dissolved (%) | | | |
|---|---|---|---|
| 3 | 2 | 1 | Time (min) |
| 0 | 0 | 0 | 0 |
| 0 | 0 | 0 | 15 |
| 0 | 0 | 0 | 30 |
| 0 | 0 | 0 | 45 |
| 0 | 0 | 0 | 60 |
| 0 | 8 | 0 | 75 |
| 93 | 92 | 90 | 90 |
| 99 | 97 | 96 | 105 |
| 103 | 99 | 98 | 120 |
| 103 | 101 | 99 | 240 |

6-MP release is observed after 75-90 minutes.

Enteric Coating of MCC Based 6-MP Tablets

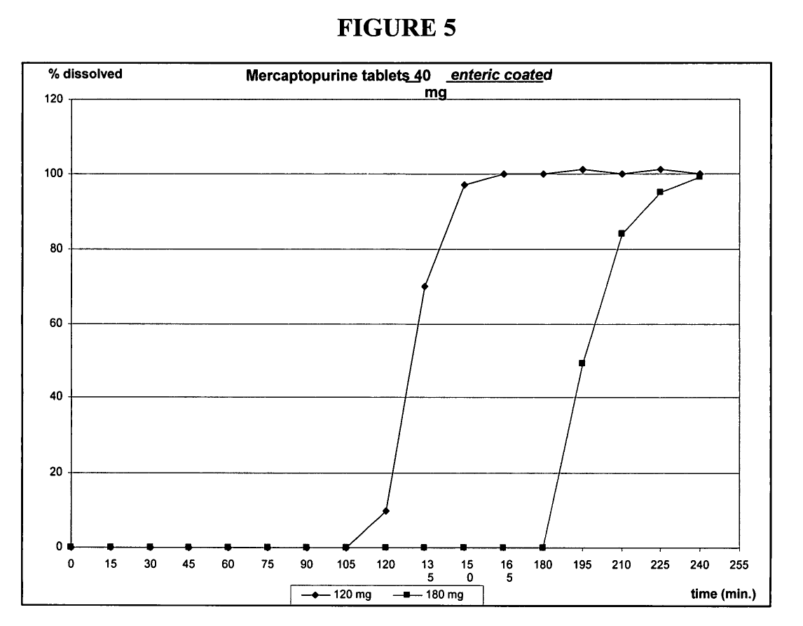

Enteric coating of the MCC based 6-MP tablets was carried out as above. Part of the batch was coated with a layer of 120 mg EUDRAGIT® L (batch ID 131.038), the other part with 180 mg of EUDRAGIT® L (batch ID 131.038.1) per tablet. The dissolution results of these tablets are shown in FIG. 5.

FIG. 5 shows that a coating of 120 mg EUDRAGIT® L per tablet gives a delay of about two hours in intestinal buffer while a coating of 180 mg gives a delay of about 3 hours in the same buffer.

What is claimed is:

1. An enterically coated pharmaceutical composition comprising 6-mercaptopurine wherein the enteric coating imparts a delay in the release of the 6-mercaptopurine following oral administration of the pharmaceutical composition such that release of 6-mercaptopurine occurs after passage of the composition through the stomach.

2. An enterically coated pharmaceutical composition comprising 6-mercaptopurine and a potassium, sodium, magnesium, ammonium, or calcium salt of a pharmaceutically acceptable acid wherein the enteric coating imparts a delay in the release of the 6-mercaptopurine following oral administration of the pharmaceutical composition such that substantially no release of 6-mercaptopurine occurs before passage of the composition through the stomach.

3. The enterically coated pharmaceutical composition of claim 1 further comprising a delay coating which imparts a further delay in the release of the 6-mercaptopurine such that substantially no release of 6-mercaptopurine occurs until a predetermined period of time after passage of the composition through the stomach.

4. The enterically coated pharmaceutical composition of claim 3 wherein the predetermined period of time is at least about one hour, at least about two hours, or at least about three hours.

5. The pharmaceutical composition of claim 3 wherein the pharmaceutically acceptable acid is selected from the group consisting of acetic acid, ascorbic acid, benzoic acid, citric acid, and tartaric acid.

6. The pharmaceutical composition of claim 3 comprising potassium citrate.

7. The pharmaceutical composition of claim 3 wherein the 6-mercaptopurine was spray granulated from a solution onto a pharmaceutical carrier powder to form a uniform coating of 6-mercaptopurine over the pharmaceutical carrier powder.

8. The pharmaceutical composition of claim 7 wherein the spray granulation was carried out in a fluidized bed.

9. The pharmaceutical composition of claim 7 wherein the solution of 6-mercaptopurine comprises:
(a) a solvent selected from the group consisting of dimethylformamide, dimethylacetamide, dimethylsulfoxide, and mixtures thereof; or
(b) a solvent selected from the group consisting of: water and an at least about stoichiometric amount of a pharmaceutically acceptable base, ethanol and an at least about stoichiometric amount of a pharmaceutically acceptable base, and ethanol/water mixtures and an at least about stoichiometric amount of a pharmaceutically acceptable base.

10. The pharmaceutical composition of claim 9 wherein the solvent comprises ethanol/water/potassium hydroxide, ethanol/water/sodium hydroxide, or ethanol/potassium hydroxide.

11. The pharmaceutical composition of claim 7 wherein the pharmaceutical carrier powder comprises a powder selected from the group consisting of: lactose, starch, microcrystalline cellulose, calcium phosphate, powdered cellulose, sorbitol, and sucrose.

12. The pharmaceutical composition of claim 11 wherein the pharmaceutical carrier powder comprises lactose or microcrystalline cellulose.

13. The pharmaceutical composition of claim 10 wherein the pharmaceutical carrier powder is pre-sprayed with a solution of a pharmaceutically acceptable acid in a molar amount that was greater than the molar amount of potassium hydroxide in the 6-mercaptopurine solution applied to the pharmaceutical carrier powder.

14. The pharmaceutical composition of claim 13 wherein the acid is selected from the group consisting of: acetic acid, ascorbic acid, benzoic acid, citric acid, and tartaric acid.

15. The pharmaceutical composition of claim 14 wherein the acid is citric acid.

16. A pharmaceutical composition comprising 6-mercaptopurine wherein:
(a) the dissolution rate of the 6-mercaptopurine is greater than 50% within seven minutes when measured in 900 ml of 0.1N HCl at 37° C. in a USP type II device using paddles rotating at 50 rpm;
(b) the time to reach 50% dissolution of the 6-mercaptopurine is reduced by at least about 30% compared to the standard formulation when measured in 900 ml of 0.1N HCl at 37° C. in a USP type II device using paddles rotating at 50 rpm; or
c) the bioavailability of the 6-mercaptopurine is improved by at least about 15% when the pharmaceutical composition is dosed to a mammal as compared to the standard formulation;
wherein the pharmaceutical composition is coated with an enteric coating that imparts a delay in the release of the 6-mercaptopurine following oral administration of the pharmaceutical composition such that release of 6-mercaptopurine occurs after passage of the composition through the stomach.

17. The pharmaceutical composition of claim 16 wherein substantially no release of 6-mercaptopurine occurs before passage of the composition through the stomach.

18. The pharmaceutical composition of claim 16 further comprising a delay coating which imparts a further delay in the release of the 6-mercaptopurine such that substantially no release of 6-mercaptopurine occurs until a predetermined period of time after passage of the composition through the stomach.

19. The pharmaceutical composition of claim 18 wherein the predetermined period of time is at least about one hour, at least about two hours, or at least about three hours.

20. The pharmaceutical composition of claim 16 wherein the dissolution of the 6-mercaptopurine in the non-coated pharmaceutical composition is greater than 50% within five minutes when measured in 900 ml of 0.1N HCl at 37° C. in a USP type II device using paddles rotating at 50 rpm.

21. The pharmaceutical composition of claim 16 wherein the 6-mercaptopurine forms a uniform coating over a pharmaceutical carrier powder.

22. The pharmaceutical composition of claim 21 wherein the 6-mercaptopurine was spray granulated from a solution onto the pharmaceutical carrier powder to form a uniform coating of 6-mercaptopurine over the pharmaceutical carrier powder.

23. The pharmaceutical composition of claim 22 wherein the spray granulation was carried out in a fluidized bed.

24. The pharmaceutical composition of claim 22 wherein the solution of 6-mercaptopurine comprises:
(a) a solvent selected from the group consisting of dimethylformamide, dimethylacetamide, dimethylsulfoxide, and mixtures thereof; or
(b) a solvent selected from the group consisting of: water and an at least about stoichiometric amount of a pharmaceutically acceptable base, ethanol and an at least about stoichiometric amount of a pharmaceutically acceptable base, and ethanol/water mixtures and an at least about stoichiometric amount of a pharmaceutically acceptable base.

25. The pharmaceutical composition of claim 24 wherein the solvent comprises ethanol/water/potassium hydroxide, ethanol/water/sodium hydroxide, or ethanol/potassium hydroxide.

26. The pharmaceutical composition of claim 22 wherein the pharmaceutical carrier powder comprises a powder selected from the group consisting of: lactose, starch, microcrystalline cellulose, calcium phosphate, powdered cellulose, sorbitol, and sucrose.

27. The pharmaceutical composition of claim 26 wherein the pharmaceutical carrier powder comprises lactose or microcrystalline cellulose.

28. The pharmaceutical composition of claim 22 wherein the 6-mercaptopurine was spray granulated from a solution containing potassium hydroxide onto the pharmaceutical carrier powder and wherein the pharmaceutical carrier powder was pre-sprayed with a solution of a pharmaceutically acceptable acid in a molar amount that was at least about slightly greater than the molar amount of potassium hydroxide in the 6-mercaptopurine solution spray granulated onto the pharmaceutical carrier powder.

29. The pharmaceutical composition of claim 28 wherein the acid was selected from the group consisting of: acetic acid, ascorbic acid, benzoic acid, citric acid, and tartaric acid.

30. The pharmaceutical composition of claim 29 wherein the acid was citric acid.

31. The pharmaceutical composition of claim 16 wherein the improved bioavailability is a rise in average AUCt or AUC, of about 5%, or about 15%, or about 20%.

32. The pharmaceutical composition of claim 16 wherein the improved bioavailability is a rise in the average ratio of the individual AUCt values for the non-coated pharmaceutical composition as compared to the standard formulation of about 1.05, or about 1.15, or about 1.20.

33. The pharmaceutical composition of claim 16 wherein the dissolution of a tablet comprising the pharmaceutical composition is measured in part (a) or part (b).

34. The pharmaceutical composition of claim 33 wherein the tablet comprises 50 mg of 6-mercaptopurine.

35. A pharmaceutical composition comprising about 3% to about 20% of 6-mercaptopurine and about 2% to about 30% of potassium citrate wherein the composition exhibits enhanced solubility in aqueous acid as compared to the standard formulation;
and wherein the pharmaceutical composition is coated with an enteric coating that imparts a delay in the release of the 6-mercaptopurine following oral administration of the pharmaceutical composition such that release of 6-mercaptopurine occurs after passage of the composition through the stomach.

36. The pharmaceutical composition of claim 35 wherein substantially no release of 6-mercaptopurine occurs before passage of the composition through the stomach.

37. The pharmaceutical composition of claim 35 further comprising a delay coating which imparts a further delay in the release of the 6-mercaptopurine such that substantially no release of 6-mercaptopurine occurs until a predetermined period of time after passage of the composition through the stomach.

38. The pharmaceutical composition of claim 37 wherein the predetermined period of time is at least about one hour, at least about two hours, or at least about three hours.

39. The pharmaceutical composition of claim 38 comprising about 8% 6-mercaptopurine and about 5% potassium citrate.

40. The pharmaceutical composition of claim 39 wherein:
(a) the 6-mercaptopurine was spray granulated from solution onto the pharmaceutical carrier powder; and
(b) the pharmaceutical carrier powder was pre-sprayed with a solution of citric acid; so that the 6-mercaptopurine forms a uniform coating on the pharmaceutical carrier powder.

41. A composition of matter comprising 1 to 35% 6-mercaptopurine (w/w) in a uniform coating on the surface of a lactose granulate;
wherein the composition of matter is coated with an enteric coating that imparts a delay in the release of the 6-mercaptopurine following oral administration of the composition of matter such that release of 6-mercaptopurine occurs after passage of the composition of matter through the stomach.

42. The composition of matter of claim 41 wherein substantially no release of 6-mercaptopurine occurs before passage of the composition through the stomach.

43. The composition of matter of claim 41 further comprising a delay coating which imparts a further delay in the release of the 6-mercaptopurine such that substantially no release of 6-mercaptopurine occurs until a predetermined period of time after passage of the composition of matter through the stomach.

44. The composition of matter of claim 43 wherein the predetermined period of time is at least about one hour, at least about two hours, or at least about three hours.

45. A method of making a pharmaceutical composition comprising 6-mercaptopurine, the method comprising coating a solution of 6-mercaptopurine onto a pharmaceutical carrier so that the 6-mercaptopurine forms a uniform coating on the pharmaceutical carrier, thereby forming a pharmaceutical composition comprising 6-mercaptopurine that exhibits enhanced 6-mercaptopurine solubility properties in aqueous acid as compared to the standard formulation;
and coating the pharmaceutical composition with an enteric coating that imparts a delay in the release of the 6-mercaptopurine following oral administration of the pharmaceutical composition such that release of 6-mercaptopurine occurs after passage of the pharmaceutical composition through the stomach.

46. The method of claim 45 wherein substantially no release of 6-mercaptopurine occurs before passage of the composition through the stomach.

47. The method of claim 45 further comprising providing a delay coating under the enteric coating wherein the delay coating imparts a further delay in the release of the 6-mercaptopurine such that substantially no release of 6-mercaptopurine occurs until a predetermined period of time after passage of the pharmaceutical composition through the stomach.

48. The method of claim 47 wherein the predetermined period of time is at least about one hour, at least about two hours, or at least about three hours.

49. The method of claim 45 comprising spray granulating the solution of 6-mercaptopurine onto the pharmaceutical carrier.

50. The method of claim 49 wherein the 6-mercaptopurine was spray granulated from a solution containing potassium hydroxide onto the pharmaceutical carrier powder and wherein the pharmaceutical carrier is pre-sprayed with a solution of a pharmaceutically acceptable acid in a molar amount that is at least about slightly greater than the molar amount of potassium hydroxide in the 6-mercaptopurine solution applied to the pharmaceutical carrier.

51. The method of claim 50 wherein the spray granulation uses a fluidized bed granulator.

52. The method of claim 45 wherein the 6-mercaptopurine in the non-coated pharmaceutical composition dissolves in 0.1N HCl to an extent of greater than 50% within seven minutes.

53. The method of claim 45 wherein the time to reach 50% dissolution of the 6-mercaptopurine in the non-coated pharmaceutical composition is reduced by at least about 30% compared to the standard formulation when measured in 900 ml of 0.1N HCl at 37° C. in a USP type II device using paddles rotating at 50 rpm.

54. The method of claim 53 wherein the dissolution of a tablet comprising the non-coated pharmaceutical composition is measured.

55. The method of claim 54 wherein the tablet comprises 50 mg of 6-mercaptopurine.

56. The method of claim 51 wherein the solution of 6-mercaptopurine comprises:
(a) a solvent selected from the group consisting of dimethylformamide, dimethylacetamide, dimethylsulfoxide, and mixtures thereof; or
(b) a solvent selected from the group consisting of: water and an at least about stoichiometric amount of a pharmaceutically acceptable base, ethanol and an at least about stoichiometric amount of a pharmaceutically acceptable base, and ethanoVwater mixtures and an at least about stoichiometric amount of a pharmaceutically acceptable base.

57. The method of claim 56 wherein the solvent is selected from the group consisting of: ethanol/water/potassium hydroxide, ethanol/water/sodium hydroxide, and ethanol/potassium hydroxide.

58. The method of claim 56 wherein the solvent consists essentially of: dimethylformamide, dimethylacetamide, dimethylsulfoxide, or mixtures thereof; ethanol/water/potassium hydroxide, ethanol/water/sodium hydroxide, or ethanol/potassium hydroxide.

59. The method of claim 56 wherein the pharmaceutical carrier comprises a powder selected from the groups consisting of: lactose, starch, microcrystalline cellulose, calcium phosphate, powdered cellulose, sorbitol, and sucrose.

60. The method of claim 59 wherein the pharmaceutical carrier comprises a lactose powder or microcrystalline cellulose.

61. The method of claim 47 wherein the bioavailability of the 6-mercaptopurine is improved by at least about 15% as compared to the standard formulation when dosing said non-coated composition to a mammal.

62. A method of dosing 6-mercaptopurine to patients in need of treatment with 6-mercaptopurine comprising administering an enterically coated pharmaceutical composition comprising 6-mercaptopurine to patients wherein the 6-mercaptopurine is released after a delay of at least one hour after the enterically coated pharmaceutical composition leaves the stomach.

63. A method of dosing a pharmaceutical composition comprising 6-mercaptopurine to patients in need of treatment with 6-mercaptopurine comprising administering a pharmaceutical composition comprising 6-mercaptopurine to patients wherein:
(a) the pharmaceutical composition displays enhanced 6-mercaptopurine solubility in aqueous acid compared to the standard formulation; or
(b) the bioavailability of the 6-mercaptopurine in the pharmaceutical composition is improved by at least 15% when the pharmaceutical composition is dosed to a mammal as compared to the standard formulation;
and wherein the pharmaceutical composition is coated with an enteric coating that imparts a delay in the release of the 6-mercaptopurine following oral administration of the pharmaceutical composition such that release of 6-mercaptopurine occurs after passage of the pharmaceutical composition through the stomach.

64. The method of claim 63 wherein substantially no release of 6-mercaptopurine occurs before passage of the composition through the stomach.

65. The method of claim 63 wherein the pharmaceutical composition further comprises a delay coating under the enteric coating wherein the delay coating imparts a further delay in the release of the 6-mercaptopurine such that substantially no release of 6-mercaptopurine occurs until a predetermined period of time after passage of the pharmaceutical composition through the stomach.

66. The method of claim 64 wherein the predetermined period of time is at least about one hour, at least about two hours, or at least about three hours.

67. The method of claim 63 wherein the 6-mercaptopurine in the non-coated pharmaceutical composition dissolves in 0.1N HCl to an extent of greater than 50% within seven minutes.

68. The method of claim 63 wherein the time to reach 50% dissolution of the 6-mercaptopurine in the non-coated pharmaceutical composition is reduced by at least about 30% compared to the standard formulation when measured in 900 ml of 0.1N HCl at 37° C. in a USP type II device using paddles rotating at 50 rpm.

69. The method of claim 68 wherein the dissolution of a tablet comprising the non-coated pharmaceutical composition is measured.

70. The method of claim 69 wherein the tablet comprises 50 mg of 6-mercaptopurine.

71. The method of claim 65 wherein the mammal is at least one of the patients.

72. A method of treating leukemia or other cancers, Crohn's disease, arthritis, or ulcertative colitis comprising administering an enterically coated pharmaceutical composition comprising 6-mercaptopurine to a patient having or suspected of having leukemia or another cancer, Crohn's disease, arthritis, or ulcerative colitis wherein the 6-mercaptopurine is released after a delay of at least one hour after the enterically coated pharmaceutical composition leaves the stomach.

73. A method of treating leukemia or other cancers, Crohn's disease, arthritis, or ulcertative colitis comprising administering a pharmaceutical composition comprising 6-mercaptopurine to a patient having or suspected of having leukemia or another cancer, Crohn's disease, arthritis, or ulcerative colitis wherein:
(a) the pharmaceutical composition displays enhanced 6-mercaptopurine solubility in aqueous acid compared to the standard formulation;
(b) the bioavailability of the 6-mercaptopurine in the pharmaceutical composition is improved by at least 15% when the pharmaceutical composition is dosed to a mammal as compared to the standard formulation; or
(c) the dose of the 6-mercaptopurine in the pharmaceutical composition is reduced by at least 15% as compared to the standard formulation yet achieves the same bioavailability as the standard formulation;
wherein the pharmaceutical composition is coated with an enteric coating that imparts a delay in the release of the 6-mercaptopurine following oral administration of the pharmaceutical composition such that release of 6-mercaptopurine occurs after passage of the pharmaceutical composition through the stomach.

74. The method of claim 71 wherein substantially no release of 6-mercaptopurine occurs before passage of the composition through the stomach.

75. The method of claim 74 wherein the pharmaceutical composition further comprises a delay coating under the enteric coating wherein the delay coating imparts a further delay in the release of the 6-mercaptopurine such that substantially no release of 6-mercaptopurine occurs until a predetermined period of time after passage of the pharmaceutical composition through the stomach.

76. The method of claim 75 wherein the predetermined period of time is at least about one hour, at least about two hours, or at least about three hours.

77. The method of claim 72 wherein the leukemia is acute lymphocytic leukemia.

78. The method of claim 73 wherein the 6-mercaptopurine in the non-coated pharmaceutical composition dissolves in 0.1N HCl to an extent of greater than 50% within seven minutes.

79. The method of claim 73 wherein the time to reach 50% dissolution of the 6-mercaptopurine in the non-coated pharmaceutical composition is reduced by at least about 30% compared to the standard formulation when measured in 900 ml of 0.1N HCl at 37° C. in a USP type II device using paddles rotating at 50 rpm.

80. The method of claim 79 wherein the dissolution of a tablet comprising the non-coated pharmaceutical composition is measured.

81. The method of claim 80 wherein the tablet comprises 50 mg of 6-mercaptopurine.

82. The method of claim 75 wherein the mammal is the patient.

83. A granulate for preparing a dosage form of 6-mercaptopurine comprising a particle of a pharmaceutical carrier powder coated with 6-mercaptopurine wherein the granulate is coated with an enteric coating that imparts a delay in the release of the 6-mercaptopurine following oral administration of the granulate such that release of 6-mercaptopurine occurs after passage of the granulate through the stomach.

84. The granulate of claim 83 wherein substantially no release of 6-mercaptopurine occurs before passage of the composition through the stomach.

85. The granualte of claim 83 wherein the granulate further comprises a delay coating under the enteric coating wherein the delay coating imparts a further delay in the release of the 6-mercaptopurine such that substantially no release of 6-mercaptopurine occurs until a predetermined period of time after passage of the granulate through the stomach.

86. The granulate of claim 85 wherein the predetermined period of time is at least about one hour, at least about two hours, or at least about three hours.

87. The granulate of claim 83 wherein the pharmaceutical carrier powder comprises a powder selected from the groups consisting of: lactose, starch, microcrystalline cellulose, calcium phosphate, powdered cellulose, sorbitol, and sucrose.

88. The granulate of claim 87 wherein the pharmaceutical carrier powder comprises a powder of lactose or microcrystalline cellulose.

89. A pharmaceutical dosage form comprising:
a core comprising 6-mercaptopurine; and
an enteric coating;
wherein the enteric coating imparts a delay in the release of the 6-mercaptopurine following oral administration of the dosage form such that release of 6-mercaptopurine occurs after passage of the dosage form through the stomach.

90. The pharmaceutical dosage form of claim 89 wherein the core comprises:
(a) 6-mercaptopurine and a potassium, sodium, magnesium, ammonium, or calcium salt of a pharmaceutically acceptable acid; or
(b) a uniform coating of 6-mercaptopurine over a pharmaceutical carrier powder.

91. The pharmaceutical dosage form of claim 89 wherein the core has the following characteristics:
(a) the dissolution rate of the 6-mercaptopurine is greater than 50% within seven minutes when measured in 900 ml of 0.1N HCl at 37° C. in a USP type II device using paddles rotating at 50 rpm;
(b) the time to reach 50% dissolution of the 6-mercaptopurine is reduced by at least about 30% compared to the standard formulation when measured in 900 ml of 0.1N HCl at 37° C. in a USP type II device using paddles rotating at 50 rpm; or
c) the bioavailability of the 6-mercaptopurine is improved by at least about 15% when the core is dosed to a mammal as compared to the standard formulation.
